SEQUENCE LISTING (23 sequences)

SEQ ID NO: 1 - DNA, 888 bp, Canis familiaris
atggaaaccgacaccctgctgctgtgggtgctgctcctgtgggtccccggcagcaccggcagcctgcggcggagcagctgcttcggcggcaggatggaccggatcggcgcccagagcggcctgggctgcaacagcttcagatacggcggcagcggcggctctggcggaagcggaggatccgggggcagcggaggaagcggcggctgcaccgacacccccccctgccccgtgcctgagcccctgggcggacccagcgtgctgatcttcccccccaagcccaaggacatcctgcggatcacccggacccccgaggtgacctgcgtggtgctggacctgggcagagaagatcccgaggtgcagatcagctggttcgtggacggcaaagaggtgcacaccgccaagacccagagccgggagcagcagttcaacggcacctaccgggtggtgtccgtgctgcccatcgagcaccaggactggctgaccggcaaagaattcaagtgccgggtgaaccacatcgacctgcccagccccatcgagcggaccatcagcaaggccagaggcagggcccacaagcccagcgtgtacgtgctgcctcccagccccaaagagctgtccagcagcgacaccgtgagcatcacctgcctgatcaaggacttctacccccccgacatcgacgtggagtggcagagcaacggccagcaggaacccgagcggaagcaccggatgaccccccctcagctggacgaggacggcagctacttcctgtacagcaagctgtccgtggacaagagccggtggcagcagggcgaccccttcacctgcgccgtgatgcacgagacactgcagaaccactacaccgacctgagcctgagccacagccccggcaag

SEQ ID NO: 2 - protein, 296 aa, Canis familiaris
METDTLLLWVLLLWVPGSTGSLRRSSCFGGRMDRIGAQSGLGCNSFRYGGSGGSGGSGGSGGSGGSGGCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 3 - protein, 276 aa, Canis familiaris
SLRRSSCFGGRMDRIGAQSGLGCNSFRYGGSGGSGGSGGSGGSGGSGGCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 4 - DNA, 876 bp, Canis familiaris
atggaaaccgacaccctgctgctgtgggtgctgctgctctgggtccccggcagcaccggcagcctgcggcggagcagctgcttcggcggcaggatggaccggatcggcgcccagagcggcctgggctgcaacagcttcagatacggcggcagcggcggctctggcggaagcggaggatccgggggcagcggaggaagcggcggctgccccaagtgccctgcccccgagatgctgggcggacccagcgtgttcatcttcccccccaagcccaaggataccctgctgatcgcccggacccccgaggtgacctgcgtggtggtggacctggaccccgaggaccctgaggtgcagatcagctggttcgtggacggcaagcagatgcagaccgccaagacccagccccgggaggaacagttcaacggcacctaccgggtggtgtccgtgctgcccatcggccaccaggactggctgaagggcaagcagtttacctgtaaggtcaacaacaaggccctgcccagccccatcgagcggaccatcagcaaggccagaggccaggcccatcagcccagcgtgtacgtgctgcctcccagcagagaggaactgagcaagaacaccgtgagcctgacctgcctgatcaaggacttcttcccccctgacatcgacgtggagtggcagagcaacggccagcaggaacccgagagcaagtaccggaccaccccccctcagctggacgaggacggcagctacttcctgtacagcaagctgtccgtggacaagagccggtggcagcggggcgacaccttcatctgcgccgtgatgcacgaggccctgcacaaccactacacccaggaaagcctgagccacagccccggcaag

SEQ ID NO: 5 - protein, 292 aa, Canis familiaris
METDTLLLWVLLLWVPGSTGSLRRSSCFGGRMDRIGAQSGLGCNSFRYGGSGGSGGSGGSGGSGGSGGCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 6 - protein, 272 aa, Canis familiaris
SLRRSSCFGGRMDRIGAQSGLGCNSFRYGGSGGSGGSGGSGGSGGSGGCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 7 - protein, 20 aa, Mus musculus
METDTLLLWVLLLWVPGSTG

SEQ ID NO: 8 - protein, 28 aa, Canis familiaris
SLRRSSCFGGRMDRIGAQSGLGCNSFRY

SEQ ID NO: 9 - protein, 228 aa, Canis familiaris
CTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 10 - protein, 224 aa, Canis familiaris
CPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 11 - protein, 5 aa, CANIS FAMILIARIS
CPPCP

SEQ ID NO: 12 - protein, 20 aa, Artificial Sequence
  Synthetic - Linker
GGSGGSGGSGGSGGSGGSGG

SEQ ID NO: 13 - protein, 9 aa, Artificial Sequence
  Synthetic - Linker
GSGGSGGSG

SEQ ID NO: 14 - protein, 11 aa, Artificial Sequence
  Synthetic - Linker
GGSGGSGGSGG

SEQ ID NO: 15 - protein, 14 aa, Artificial Sequence
  Synthetic - Linker
GGSGGSGGSGGSGG

SEQ ID NO: 16 - protein, 16 aa, Artificial Sequence
  Synthetic - Linker
GSGGSGGSGGSGGSGG

SEQ ID NO: 17 - protein, 16 aa, Artificial Sequence
  Synthetic - Linker
GGSGGSGGSGGSGGSG

SEQ ID NO: 18 - protein, 8 aa, Canis familiaris
CTDTPPCP

SEQ ID NO: 19 - protein, 5 aa, Canis familiaris
CPKCP

SEQ ID NO: 20 - protein, 13 aa, CANIS FAMILIARIS
FNECRCTDTPPCP

SEQ ID NO: 21 - protein, 18 aa, Canis familiaris
PKRENGRVPRPPDCPKCP

SEQ ID NO: 22 - protein, 20 aa, CANIS FAMILIARIS
AKECECKCNCNNCPCPGCGL

SEQ ID NO: 23 - protein, 13 aa, Canis familiaris
PKESTCKCISPCP

(19) United States
(12) Patent Application Publication
(10) Pub. No.: US 2012/0093814 A1
(43) Pub. Date: Apr. 19, 2012

(54) Fusion Proteins Comprising Canine FC Portions

(75) Inventors: Keith Canada, Ridgefield, CT (US); Sanjaya Singh, Ridgefield, CT (US); Xiangyang Zhu, Ridgefield, CT (US)

(73) Assignee: BOEHRINGER INGELHEIM INTERNATIONAL GMBH, Ingelheim (DE)

(21) Appl. No.: 13/254,887

(22) PCT Filed: Mar. 30, 2010
(86) PCT No.: PCT/US10/29151

§ 371 Date: Dec. 21, 2011

Related U.S. Application Data
(60) Provisional application No. 61/164,616, filed on Mar. 30, 2009.

(51) Int. Cl. A61K 39/395; A61P 9/12; A61P 9/00; C07K 16/00; C07H 21/04

(52) U.S. Cl. 424/134.1; 530/387.3; 536/23.5

(57) ABSTRACT

The present invention relates to therapeutic peptides and proteins fused to a canine antibody Fc domain. Methods and compositions of using the same are described.

### FIELD OF THE INVENTION

The present invention relates to therapeutic peptides and proteins fused to a canine antibody Fc domain. Methods and compositions of using the same are described.

### BACKGROUND OF THE INVENTION

Use of Fc fusion proteins is now a common modality for increasing the half life of given therapeutic agent in vivo. It is recognized that humans have four human IgG subclasses (namely IgG1, IgG2, IgG3 and IgG4) and the Fc domains of each of these IgGs have been variously employed as part of potential therapeutic proteins. Antibody “effector functions” are biological activities attributable to an Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; down regulation of cell surface receptors (e.g. B cell receptor; BCR), etc.

These effector functions of an antibody tend to vary depending on the native IgG subclass. For example, it is known that IgG the ADCC toxicity response to human Fc domains varies depending on the particular subclass of IgG. For example, IgG1 and IgG3 are known to induce a much larger ADCC response than IgG2 and IgG4 has lower ADCC response than IgG2-based therapeutics. It is recognized that it is desirable to minimize the ADCC toxicity response from the Fc fusion.

When the phylogenetic tree of the constant regions of canine, human and mouse IgG y-chains are compared, it becomes readily apparent that although there is significant sequence homology in the constant regions of subclasses within a species, there is very little homology when the constant regions are compared across species (Teng et al. Vet. Immunol. Immunopath. 80 2001, 259-270; see FIG. 4 therein).

Given that the Fc domain has important effector functions it is important to determine that a given therapeutic response of an Fc-fusion based therapeutic protein is attributable to the therapeutic protein as opposed to the Fc portion of the fusion protein. For agents that are tested in animals, it would be beneficial to characterize the fusion protein response based on Fc portions that are derived from IgGs of the species in which the response is being monitored. This is particularly important when a therapeutic agent is being tested in trials and for dosing regimens. In this regard, there is a need to create specific Fc domains that can be used for testing and treatment of a given therapeutic composition in dogs.

### BRIEF SUMMARY OF THE INVENTION

The invention described herein relates to a fusion protein comprising a therapeutic peptide or protein and a canine antibody Fc domain wherein the therapeutic peptide or protein is linked to the Fc domain directly or through a linker, wherein the Fc domain comprises a hinge region having a sequence selected from the group consisting of the hinge region of a canine IgG selected from the group consisting of canine IgGA, canine IgGB, canine IgGC and canine IgGD.

Preferably, the fusion protein of claim 1, wherein the fusion protein comprises the following formula: X-La-F:F-La-X or X-La-F:F, wherein,

X is a therapeutic peptide; L is a linker comprising a amino acid residues; a is an integer of at least 0; “:” is a chemical association or crosslink; and F is at least a portion of a canine immunoglobulin Fc domain comprising an FcRn binding site and comprises a hinge region selected from an canine IgGA, canine IgGB, canine IgGC and canine IgGD.

In specific embodiments, the canine hinge region comprises a sequence selected from the group consisting of CTDTPPCP (SEQ ID NO:18); CPKCP (SEQ ID NO:19); FNECRCTDTPPCP (SEQ ID NO:20); PKRENGRVPRPPDCPKCP (SEQ ID NO:21); AKECECKCNCNNCPCPGCGL (SEQ ID NO:22); and PKESTCKCISPCP (SEQ ID NO:23).

The therapeutic protein may be any therapeutic protein that is to be delivered as an Fc fusion construct, exemplary embodiments contemplate the therapeutic peptide is a natiuretic peptide selected from the group consisting of ANP, BNP, Urodilatin, DNP or a biologically active sequence variant thereof. Specific embodiments describe the therapeutic peptide as ANP or BNP. The fusion protein can comprise at least two therapeutic peptides. Both peptides may be the same or may be different peptides. In specific examples at least one of peptides is a natiuretic peptide, preferably having the sequence of SEQ ID NO:8.

In specific embodiments, the fusion protein comprises at least two Fc domains. The Fc domains are from canine IgGA, IgGB, IgGC or IgGD. The two Fc domains may both be from the same subclass of IgG or from different subclasses. Thus the first Fc domain may be from IgGA, IgGB, IgGC or IgGD and the second Fc domain is independently selected from IgGA, IgGB, IgGC or IgGD, it is preferably the same as the first Fc domain but may be different.

The linker in the fusions of the invention may be of any length preferably it is 6 amino acids in length, 11 amino acids in length, 16 amino acids in length or 20 amino acids in length. Other embodiments contemplate linkers that are 6 to 11 amino acids in length, 11 to 16 amino acids in length, 16 to 20 amino acids in length, 16 to 25 amino acids in length or 20 to 30 amino acids in length. Specific embodiments contemplate a linker that is a glycine succinate linker, an amino acid linker or combination thereof.

The amino acid amino acid linker may be GlyGly (L2), Gly(SerGlyGly)2SerGly (L3) (SEQ ID NO. 13), (GlyGlySer)3 GlyGly (L4) (SEQ ID NO. 14), (GlyGlySer)4GlyGly (SEQ ID NO. 15), (GlySerGly)5Gly (L5a) (SEQ ID NO. 16), (GlyGlySer)5Gly (L5) (SEQ ID NO. 17), or (GlyGlySer)6GlyGly (L6) (SEQ ID NO:12).

Also contemplated is a fusion protein comprising at least one or more therapeutic peptide (such as a peptide having a sequence of SEQ ID NO:8) separated from each other by a canine antibody Fc domain that comprises a hinge region selected from the group consisting of CTDTPPCP (SEQ ID NO:18); CPKCP (SEQ ID NO:19); FNECRCTDTPPCP (SEQ ID NO:20); PKRENGRVPRPPDCPKCP (SEQ ID NO:21); AKECECKCNCNNCPCPGCGL (SEQ ID NO:22); and PKESTCKCISPCP (SEQ ID NO:23), wherein said therapeutic peptides are conjugated to the Fc domain directly or through a linker. The fusion protein preferably has the formula:

X-La-F:F-La-X, wherein

X is one or more therapeutic peptides

L is a linker comprising amino acid residues; a is an integer of at least 0;

“:” is a chemical association or crosslink; and

F is at least a portion of an immunoglobulin Fc domain comprising an FcRn binding site and a hinge region selected from the group consisting of CTDTPPCP (SEQ ID NO:18); CPKCP (SEQ ID NO:19); FNECRCTDTPPCP (SEQ ID NO:20); PKRENGRVPRPPDCPKCP (SEQ ID NO:21); AKECECKCNCNNCPCPGCGL (SEQ ID NO:22); and PKESTCKCISPCP (SEQ ID NO:23). In specific examples, at least one of the therapeutic peptides has a sequence of SEQ ID NO:8. In specific embodiments, X is more than one natriuretic peptide.

Also contemplated is an isolated fusion protein having the sequence of SEQ ID NO:2, SEQ ID NO:3; SEQ ID NO:5 or SEQ ID NO:6 and compositions comprising the same.

Another embodiment contemplates an isolated fusion protein that exhibits at least 99% sequence identity with a polypeptide having a sequence selected from the group consisting of SEQ ID NO. 2, SEQ ID NO. 3, SEQ ID NO. 5 and SEQ ID NO. 6.

A further embodiment contemplates an isolated nucleic acid molecule encoding a polypeptide comprising amino acid sequences selected from the group consisting of SEQ ID NO. 2, SEQ ID NO. 3, SEQ ID NO. 5 and SEQ ID NO. 6.

A still additional embodiment contemplates an isolated nucleic acid molecule encoding a polypeptide comprising amino acid sequences selected from the group consisting of SEQ ID NO. 1 and SEQ ID NO. 4.

The fusion proteins described herein may be recombinantly produced by employing mammalian, prokaryotic, yeast, plant, or transgenic expression systems.

Pharmaceutical compositions comprising the fusion proteins of the invention are contemplated. The pharmaceutical composition is preferably adapted for intravenous, subcutaneous or oral administration.

Methods of use of the compositions in treating various conditions also are contemplated, including treating or ameliorating a condition characterized by an excessive level of extracellular fluid; treating or ameliorating a pathological condition in which activation of the NPRA receptor confers a therapeutic benefit; treating or ameliorating a disease associated with abnormal diruretic, natriuretic and vasodilatory activity; treating or ameliorating a disease in which it is desirable to induce naturesis, diuresis, vasodilation or to modulate the renin-angiotensin II and aldosterone systems; treating or ameliorating a pathological condition of the cardiovascular system selected from the group consisting of chronic heart failure (non-ischemic), reperfusion injury, left ventricular dysfunction (LVD), cardiac fibrosis, diastolic heart failure, and hypertrophic cardiomyopathy; treating or ameliorating a hypertensive disorder selected from the group consisting of hypertension, pulmonary hypertension, systolic hypertension and resistant hypertension; and treating or ameliorating diabetic nephropathy wherein such methods comprise administering to a subject in need thereof a therapeutically effective amount of the pharmaceutical composition described herein.

### BRIEF DESCRIPTION OF SEVERAL VIEWS OF THE DRAWINGS

FIG. 1—DNA & protein sequences of recombinant canine ANP-Fc fusion proteins. Mouse IgG kappa light chain signal sequence is in bold. This is cleaved off and is NOT on the final product. CaANP28 is underlined. The (GGS)6GG linker is italicized.

FIG. 2—Canine ANP-Fc fusion A) vector map and B) example protein map.

FIG. 3—Dose response curves for canine ANP-Fc fusion protein cGMP induction assay. Solid line is ANP, dashed line is canine ANP-Fc fusion.

FIG. 4—Effect of IV and SC ANP-caFc administration on plasma ANP-caFc levels in conscious dogs.

FIG. 5—Effect of IV and SC ANP-caFc administration on plasma cGMP levels in conscious dogs.

FIG. 6—Effect of subcutaneous ANP-caFc administration on plasma cGMP levels in conscious telemeterized beagles in Study #1.

FIGS. 7A and 7B—FIG. 7A shows the effect of subcutaneous ANP-caFc administration on absolute mean arterial pressure on Day 1 post-dose, and FIG. 7B shows the absolute change in mean arterial pressure (MAP) across Days 1-4 post-dose in conscious telemeterized beagles in Study #1. ANPca-Fc dose-dependently decreased mean arterial blood pressure in conscious dogs. When blood samples were taken (↑) on Day 1 MAP increased in conscious dogs as expected.

FIG. 8A and FIG. 8B—FIG. 8A shows the effect of subcutaneous ANP-caFc administration on absolute heart rate (HR) on Day 1 post-dose, and FIG. 8B shows the absolute change in HR across Days 1-4 post-dose in conscious telemeterized beagles in Study #1. ANP-caFc dose-dependently increased HR in conscious dogs. When blood samples were taken (↑) on Day 1 HR increased in conscious dogs as expected.

FIG. 9—Effect of subcutaneous ANP-caFc administration on circulating cGMP levels in conscious telemeterized beagles in Study #2.

FIG. 10A and FIG. 10B—FIG. 10A shows the effect of subcutaneous ANP-caFc administration on absolute mean arterial pressure on Day 1 post-dose, and FIG. 10A shows the absolute change in mean arterial pressure (MAP) across Days 1-4 post-dose in conscious telemeterized beagles in Study #2. ANP-caFc dose-dependently decreased mean arterial blood pressure in conscious dogs. When blood samples were taken (↑) on Day 1 MAP increased in conscious dogs as expected.

FIG. 11A and FIG. 11B—FIG. 11A shows the effect of subcutaneous ANP-caFc administration on absolute heart rate (HR) on Day 1 post-dose, and FIG. 11B shows the absolute change in HR across Days 1-4 post-dose in conscious telemeterized beagles in Study #2. ANP-caFc dose-dependently increased HR in conscious dogs. When blood samples were taken (↑) on Day 1 HR increased in conscious dogs as expected.

### DETAILED DESCRIPTION OF THE INVENTION

The present disclosure is specifically directed to methods and compositions for making and using fusion proteins that comprise a therapeutic peptide or protein linked to a canine antibody Fc domain, either directly or through a linker. The therapeutic peptide and the Fc region of the fusion proteins serve two distinct biological roles that contribute to efficacy of the fusion proteins. Surprisingly, linker length also influences efficacy of the fusion proteins. The fusion proteins described herein will be useful in providing an indication of the therapeutic efficacy of drugs in dogs to predict the efficacy of treatment in other animals. Alternatively, the fusion proteins of the invention also may be useful in veterinary therapies for the treatment of dogs.

Also provided are fusion proteins that comprise at least two therapeutic peptides or proteins separated from each other by a canine antibody Fc domain, wherein the therapeutic peptides or proteins are conjugated to the a canine antibody Fc domain directly or through a linker. It is an important feature of the present invention that the canine Fc domain comprise a hinge region of a canine IgG. The hinge region may be from the same IgG subclass as the remaining canine Fc domain or alternatively, the hinge region may be from a canine IgG that is different from the IgG from which the remainder of the Fc portion is derived. Specific examples of sequences for the canine Fc hinge sequences used in the present invention include:

CTDTPPCP (SEQ ID NO:18; Hinge from canine IgGA); CPKCP (SEQ ID NO:19; Hinge from canine IgGB); FNECRCTDTPPCP (SEQ ID NO:20; Hinge from canine IgGA); PKRENGRVPRPPDCPKCP (SEQ ID NO:21; Hinge from canine IgGB); AKECECKCNCNNCPCPGCGL (SEQ ID NO:22; Hinge from canine IgGC); and PKESTCKCISPCP (SEQ ID NO:23; Hinge from canine IgGD).

In specific embodiments the therapeutic peptide or protein is a natriuretic peptide or protein, which can be a canine natriuretic peptide or protein or a natriuretic peptide or protein from another species.

Also provided herein are nucleic acid molecules that encode the therapeutic fusion proteins of the present invention, and expression vectors that comprise polynucleotide sequences encoding canine natriuretic fusion proteins, for uses that include treatment or amelioration of pathological conditions in which activation of the NPRA receptor confers a therapeutic benefit on the subject, including but not limited to diseases associated with abnormal diuretic, canine natriuretic and vasodilatory activity. Fusion proteins or nucleic acid molecules according to the invention may be present in compositions that include pharmaceutically acceptable excipients, carriers or diluents.

In one aspect, the present invention is directed to fusion proteins that comprise one or more therapeutic peptides bound to an Fc domain by a glycine succinate linker. As contemplated herein, when a glycine succinate linker is used to link a therapeutic peptide and a canine Fc domain, the glycine residue of the linker is linked to the N-terminus of the Fc domain and the succinate moeity is linked to the C-terminus of the therapeutic peptide, and/or an amino acid linker of various length and sequence. In relation to the linker, the length and composition are necessary to achieve prolonged efficacy of the therapeutic peptide. As contemplated herein, the therapeutic peptide may be linked to the Fc domain in different orientations. In one orientation, the C-terminus of peptide is linked to the N-terminus of the Fc domain and in another orientation, the N-terminus of the peptide is linked to the N-terminus of the Fc domain. The Fc domain exists as a homodimer of the hinge, CH2 and CH3 regions of an IgG molecule, with the Fc domain beginning at the first N-terminal cysteine residue within the IgG hinge region and the homodimer is held together by two disulfide bonds in the hinge from the cysteine residues therein.

In a further aspect, the invention comprises pharmaceutical compositions or formulation comprising pharmaceutically acceptable excipients, carriers or diluents and any of the fusion peptides described herein.

In an additional aspect, the invention is also directed to nucleic acid molecules encoding the fusion proteins disclosed herein and expression vectors expressing said proteins.

In another aspect, the invention relates to methods to treat or ameliorate pathological conditions in which activation of the NPRA receptor confers a therapeutic benefit on the subject, including, but not limited to, diseases associated with abnormal diruretic, canine natriuretic and vasodilatory activity and/or in which it is desirable to induce naturesis, diuresis, vasodilation or to modulate the renin-angiotensin II and aldosterone systems. These conditions include those that may be characterized by an excess in extracellular fluid, including, but not limited to, pulmonary edema. In a particularly preferred embodiment, the invention includes methods to treat or ameliorate pathological conditions of the cardiovascular system including, but not limited to, chronic heart failure (non-ischemic), post-MI heart failure (ischemic CHF), acute MI, reperfusion injury, left ventricular dysfunction (LVD), cardiac fibrosis, diastolic heart failure, and hypertrophic cardiomyopathy. In addition, hypertensive disorders including, but not limited to hypertension, e.g., pulmonary hypertension, systolic hypertension, resistant hypertension and other cardiovascular related diseases such as diabetic nephropathy may be treated or ameliorated by the methods of the present invention. It is also contemplated herein that the fusion proteins and pharmaceutical compositions of the present invention may provide therapeutic benefit for subjects undergoing coronary artery bypass graft surgery (CABG).

It is also contemplated herein that the invention includes the use of the fusion proteins of the present invention in the manufacture of a medicament for the treatment or amelioration of any of the pathological conditions provided above.

In some embodiments, the fusion protein comprises the following formula:

X-La-F:F-La-X or X-La-F:F, wherein,

X is a therapeutic peptide of protein such as for example, a canine natriuretic peptide having a sequence SLRRSSCFGGRMDRIGAQSGLGCNSFRY (SEQ ID NO: 8) ;

L is a linker comprising “a” amino acid residues; a is an integer of at least 0; “:” is a chemical association or crosslink; and F is at least a portion of an immunoglobulin Fc domain comprising an FcRn binding site. Preferably, the Fc domain has a sequence of CTDTPPCPVPEPLGGPSVLIFPPKPKDILRITRTPEVTCVVLDLGREDPEVQISWFVDGKEVHTAKTQSR EQQFNGTYRVVSVLPIEHQDWLTGKEFKCRVNHIDLPSPIERTISKARGRAHKPSVYVLPPSPKELSSSD TVSITCLIKDFYPPDIDVEWQSNGQQEPERKHRMTPPQLDEDGSYFLYSKLSVDKSRWQQGDPFTCAVMH ETLQNHYTDLSLSHSPGK (SEQ ID NO: 9) or has a sequence of CPKCPAPEMLGGPSVFIFPPKPKDTLLIARTPEVTCVVVDLDPEDPEVQISWFVDGKQMQTAKT QPREEQFNGTYRVVSVLPIGHQDWLKGKQFTCKVNNKALPSPIERTISKARGQAHQPSVYVLPP SREELSKNTVSLTCLIKDFFPPDIDVEWQSNGQQEPESKYRTTPPQLDEDGSYFLYSKLSVDKS RWQRGDTFICAVMHEALHNHYTQESLSHSPGK (SEQ ID NO:10)

In some embodiments, the fusion protein comprises the following formula: X-La-F:F-La-X, wherein X is one or more therapeutic peptides, wherein at least on the canine natriuretic peptides has a sequence of SEQ ID NO:8; L is a linker comprising amino acid residues; a is an integer of at least 0; “:” is a chemical association or crosslink; and F is at least a portion of an immunoglobulin Fc domain comprising an FcRn binding site (preferably having a sequence of SEQ ID NO 9 or 10). It is contemplated that the plurality of canine natriuretic peptides may in fact be multiple copies of the sequence of SEQ ID NO:8, i.e., X=(SEQ ID NO:8)ₙ:wherein n is an integer 1, 2, 3, 4, 5, 6, 7, 8, 9 10 or more. Further X may be a fragment of SEQ ID NO:10 that present in multiples i.e., X=(fragment of SEQ ID NO:10)n.

In some embodiments, the therapeutic peptide is selected from the group consisting of ANP, BNP, Urodilatin, DNP or a biologically active sequence variant thereof. In some embodiments, the canine natriuretic peptide is ANP or BNP.

In some embodiments, the fusion protein comprises at least two therapeutic peptides. In some embodiments, X may be more than one canine natriuretic peptide. In some embodiments, both canine natriuretic peptides are ANP. In some embodiments, both canine natriuretic peptides are BNP.

In certain embodiments, the chemical association, i.e., (:) is a covalent bond. In other embodiments, the chemical association, i.e., (:) is a non-covalent interaction, e.g., an ionic interaction, a hydrophobic interaction, a hydrophilic interaction, a Van der Waals interaction or a hydrogen bond.

In some embodiments, the fusion protein comprises at least two Fc domains.

In some embodiments, the linker is at least 2, 4, 6, 9, 11, 16 or 20 amino acids in length. In other embodiments, the linker is at least 0, 1, 5, 7, 8, 10, 12, 13, 14, 15, 17, 18, 19, 21, 22, 23, 24, 25, 26, 27, 28, 29 or 30 amino acids, but may optionally be longer, e.g., between 30 and 40 amino acids in length or between 40 and 50 amino acids in length. In some embodiments, the linker is selected from 6 to 11 amino acids in length, 11 to 16 amino acids in length, 9 to 20 amino acids in length, 16 to 20 amino acids in length, 16 to 25 amino acids in length, 20 to 30 amino acids in length, 25 to 35 amino acids in length, 30 to 50 amino acids in length, 30 to 40 amino acids in length or 35 to 45 amino acids in length. In some embodiments, the linker is more than 10, more than 15, more than 20, more than 25, or more than 30 amino acids in length. In some embodiments, the linker is selected from a glycine succinate linker (L1), an amino acid linker or combination thereof. In some embodiments, the amino acid linker is GlyGly (L2), Gly(SerGlyGly)2SerGly (L3), (GlyGlySer)3GlyGly (L4), (GlyGlySer)4GlyGly, (GlySerGly)5Gly (L5a), (GlyGlySer)5Gly (L5) or (GlyGlySer)6GlyGly (L6).

In some embodiments, the fusion protein is more resistant to proteolytic degradation than a corresponding wild type therapeutic peptide or protein. In some embodiments, the fusion protein displays a longer half-life than a corresponding wild type therapeutic peptide or protein. In some embodiments, the fusion protein is made by recombinant techniques, synthetic chemistry or semi-synthetic chemistry.

In specific embodiments, the present disclosure provides canine natriuretic fusion proteins that comprise any one of SEQ ID NOS. 2 or 5 (coded by SEQ ID NOS: 33-36, respectively) which have a final processed protein sequence of SEQ ID NO:3 and SEQ ID NO:6, respectively. The sequences shown in SEQ ID NO:2 and SEQ ID NO:4 comprise a Mouse IgG kappa light chain signal sequence of METDTLLLWVLLLWVPGSTG (SEQ ID NO:7), which is cleaved off and is not present on the final product.

The present disclosure provides pharmaceutical compositions comprising a canine natriuretic fusion protein as described herein. In some embodiments, the fusion protein is adapted for intravenous, subcutaneous or oral administration.

The present disclosure provides isolated nucleic acid molecules that encode a polypeptide selected from the group consisting of SEQ ID NO. 2, SEQ ID NO. 3, SEQ ID NO. 5 and SEQ ID NO. 6. The present disclosure also provides isolated nucleic acid molecules that encode a polypeptide selected from the group consisting of SEQ ID NO. 1 and SEQ ID NO. 4. In some embodiments, the fusion protein is recombinantly produced by employing mammalian, prokaryotic, yeast, plant, or transgenic expression systems.

The present disclosure provides methods for treating or ameliorating a condition characterized by an excessive level of extracellular fluid by administering to a subject in need thereof a therapeutically effective amount of the pharmaceutical composition that comprises one or more of the therapeutic fusion peptides linked to a canine Fc domain that comprises a canine Fc hinge region as described herein.

In those embodiments where the peptide is a natiuretic peptide, the present disclosure provides methods for treating or ameliorating a pathological condition in which activation of the NPRA receptor confers a therapeutic benefit by administering to a subject in need thereof a therapeutically effective amount of the pharmaceutical composition that comprises one or more of the canine natriuretic fusion peptides as described herein.

The present disclosure provides methods for treating or ameliorating a disease associated with abnormal diruretic, canine natriuretic and vasodilatory activity by administering to a subject in need thereof a therapeutically effective amount of the pharmaceutical composition that comprises one or more of the canine natriuretic fusion peptides as described herein.

The present disclosure provides methods for treating or ameliorating a disease in which it is desirable to induce naturesis, diuresis, vasodilation or to modulate the renin-angiotensin II and aldosterone systems by administering to a subject in need thereof a therapeutically effective amount of the pharmaceutical composition that comprises one or more of the canine natriuretic fusion peptides as described herein.

The present disclosure provides methods for treating or ameliorating a pathological condition of the cardiovascular system selected from the group consisting of chronic heart failure (non-ischemic), reperfusion injury, left ventricular dysfunction (LVD), cardiac fibrosis, diastolic heart failure, and hypertrophic cardiomyopathy by administering to a subject in need thereof a therapeutically effective amount of the pharmaceutical composition that comprises one or more of the canine natriuretic fusion peptides as described herein.

The present disclosure provides methods for treating or ameliorating a hypertensive disorder selected from the group consisting of hypertension, pulmonary hypertension, systolic hypertension and resistant hypertension by administering to a subject in need thereof a therapeutically effective amount of the pharmaceutical composition that comprises one or more of the canine natriuretic fusion peptides as described herein.

The present disclosure provides methods for treating or ameliorating diabetic nephropathy by administering to a subject in need thereof a therapeutically effective amount of the pharmaceutical composition that comprises one or more of the canine natriuretic fusion peptides as described herein.

### A. DEFINITIONS

Unless otherwise defined, all technical and scientific terms used herein have the same meaning as commonly understood by one of ordinary skill in the art to which the invention belongs. The terminology used in the description of the invention herein is for the purpose of describing particular embodiments only and is not intended to be limiting of the invention. Headers are used for the convenience of the reader and are also not intended to be limiting of the invention. All publications, patent applications, patents, and other references mentioned herein are incorporated herein by reference in their entirety, as are the package inserts of any branded drugs referred to herein by their brand names.

As used in the specification and the embodiments set forth herein, the following terms have the meanings indicated.

Singular forms “a”, “an”, and “the” are intended to include the plural forms as well, unless the context clearly indicates otherwise.

A “fusion protein”, e.g. a “canine natriuretic fusion protein” as the term is used herein, refers to a protein having at least two polypeptides covalently linked, in which one polypeptide comes from one protein sequence or domain and the other polypeptide comes from another protein sequence or domain. Generally, the polypeptides of a fusion protein may be linked either directly or via a covalent linker. The term (“linker”) refers to an amino acid linker, such as a polyglycine linker, or another type of chemical linker, e.g., a glycine succinate linker, a carbohydrate linker, a lipid linker, a fatty acid linker, a polyether linker, etc. The linker may consist of at least 2, 4, 6, 9, 11, 16 or 20 amino acids in length. Alternatively, the linker may consist of at least 0, 1, 5, 7, 8, 10, 12, 13, 14, 15, 17, 18, 19, 21, 22, 23, 24, 25, 26, 27, 28, 29 or 30 amino acids, but may optionally be longer, e.g., between 30 and 40 amino acids in length or between 40 and 50 amino acids in length. Amino acids are selected from the 20 naturally occurring amino acids, of either isomeric form D or L, for example, glycine, alanine, proline, asparagine, glutamine, and lysine. A linker may be made up of a majority of amino acids that are sterically unhindered, such as glycine and alanine. The linker may comprise a range of lengths amino acid residues, such as for example, 6 to 11 amino acids in length, 11 to 16 amino acids in length, 16 to 20 amino acids in length, 16 to 25 amino acids in length, 20 to 30 amino acids in length, 30 to 35 amino acids in length, 35 to 40 amino acids in length, 40 to 45 amino acids in length or 45 to 50 amino acids in length. Some of these amino acids may be glycosylated. Non-peptide linkers are also possible. For example, alkyl linkers such as —NH—(CH2)S—C(O)—, wherein s=2-20 could be used. These alkyl linkers may further be substituted by any non-sterically hindering group such as lower alkyl (e.g., C1-C6) lower acyl, halogen (e.g., Cl, Br), CN, NH2, phenyl, etc. An exemplary non-peptide linker is a PEG linker, wherein the linker has a molecular weight of 100 to 5000 kD, preferably 100 to 500 kD. The peptide linkers may be altered to form derivatives in the same manner as described above. As described in the examples herein, the preferred linker of the fusion proteins of the present invention is a stretch of amino acids with the basic repeat (GGS)x or (GGS)xGG. For example, x may be an integer from 0 to 16. Although specific orientations are described in detail herein, polypeptides forming fusion proteins may be linked C-terminus to N-terminus, although they can also be linked C-terminus to C-terminus, N-terminus to N-terminus, or N-terminus to C-terminus and the polypeptides of the fusion protein can be in any order. It is also contemplated herein that the fusion proteins of the present invention may contain two peptide fusions. For example, the fusion protein may comprise one peptide flanked by two Fc domains, e.g., Fc-Canine natriuretic Peptide-Fc, where the canine natriuretic peptide is conjugated to the Fc domains directly or through a linker. As contemplated herein, when a glycine succinate linker is used to link a canine natriuretic peptide and a Fc domain, the glycine residue of the linker is linked to the N-terminus of the Fc domain and the succinate moeity is linked to the C-terminus of the canine natriuretic peptide.

The term “protein” is used herein interchangeably with “polypeptide” and “peptide.”

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Such analogs are familiar to one of skill in the art and include, e.g., phosphorothioates and phosphoramidates. Unless otherwise indicated, a particular nucleic acid sequence also implicitly encompasses conservatively modified variants thereof (e.g., degenerate codon substitutions) and complementary sequences, as well as the sequence explicitly indicated. As used herein, the term “nucleic acid” may also be referred to as “gene”, “cDNA”, “mRNA”, “oligonucleotide”, and “polynucleotide”.

As contemplated herein, a polynucleotide sequence comprising a fusion protein of the present invention hybridizes under stringent conditions to each of the nucleotide sequences encoding each individual polypeptide of the fusion protein. The polynucleotide sequences encoding the individual polypeptides of the fusion polypeptide therefore include conservatively modified variants, polymorphic variants, alleles, mutants, subsequences, and interspecies homologs.

“Amino acid” is defined herein as any naturally occurring, artificial, or synthetic amino acid in either its L or D stereoisomeric forms, unless otherwise specified. The term “residue” is used interchangeably with the term “amino acid,” and is often designated as having a particular position in a given sequence of amino acids.

“Biologically active” refers to an agent having therapeutic or pharmacologic activity, such as an agonist, partial agonist or antagonist.

“Effective amount” as provided herein refers to a nontoxic but sufficient amount to provide the desired therapeutic effect. As will be pointed out below, the exact amount required will vary from subject to subject, depending on age, general condition of the subject, the severity of the condition being treated, the particular biologically active agent administered, and the like. An appropriate “effective” amount in any individual case may be determined by one of ordinary skill in the art by reference to the pertinent texts and literature and/or by using routine experimentation.

As used herein, the term “Fc domain” refers to that part of an antibody derived from the stem of the “Y,” and is composed of two heavy chains that each contribute two to three constant domains (depending on the class of the antibody). The Fc region binds to various cell receptors and complement proteins, and mediates different physiological effects of antibodies. As contemplated herein, the Fc domain of any antibody which displays minimal to no effector function may be used with the present invention. These include, but are not limited to, IgG1, IgG2, IgG4 but may also include any Fc domain of any antibody, the sequence of which has been altered, according to methods familiar to one of skill in the art, to possess minimal effector activity.

Alternatively, the term “Fc domain” may be described as an IgG heavy chain comprising hinge, CH2 and CH3 regions, wherein the IgG heavy chain begins at the first N-terminal cysteine residue within the hinge region and forms a homodimer with another Fc domain at the first and second N-terminal cysteine residues.

The term “Fc” is also used to describe part of the fusion proteins. In this context, Fc is an IgG heavy chain comprising hinge, CH2 and CH3 domains, the IgG heavy chain begins at the first N-terminal cysteine residue within the hinge region and forms a homodimer with another Fc at the first and second N-terminal cysteine residues. In other words, the N-terminal amino acid sequence of each chain of the Fc homodimer begins with CysProProCysPro (SEQ ID NO: 11) of the IgG hinge region and both Cys residues are disulfide bonded.

As used herein, a peptide is said to be “isolated” or “purified” when it is substantially free of cellular material or free of chemical precursors or other chemicals. The fusion peptides of the present invention can be purified to homogeneity or other degrees of purity. The level of purification will be based on the intended use. The critical feature is that the preparation allows for the desired function of the peptide, even if in the presence of considerable amounts of other components.

“Canine natriuretic peptides” as referred to herein include the mammalian canine natriuretic factors (ANP, BNP, CNP), urodilatins and peptides analogous thereto, and analogs, active fragments, degradation products, salts, variants, derivatives and combinations thereof. Specifically, canine ANP and BNP include “canine ANP28” and “canine BNP32” are specifically contemplated.

Without being limited by any particular mode of action, as referred to herein, the pharmacokinetics of a “sustained-release, or depot formulation” may be characterized as displaying an increase in bioavailability, due to FcRn binding and recycling of FcRn-bound molecules from within acidic lysosomes back to the general circulation (V. Ghetie and E. S. Ward, Annual Rev. Immunol, 18, 739-766, (2000)).

The term “semi-synthetic” as used herein refers to a process to synthesize the fusion proteins of the present invention comprising the use of both synthetic chemistry and recombinant techniques. For example, the Fc domain of the fusion proteins disclosed herein may be made recombinantly, while the canine natriuretic peptide and linker may be made synthetically.

### B. PEPTIDE MOLECULES

This invention relates to novel, biologically active fusion proteins comprised of one or more canine natriuretic peptides linked to an Fc region of IgG or other antibody from a canine source for uses that include treatment or amelioration of pathological conditions in which activation of the NPRA receptor confers a therapeutic benefit on the subject, including but not limited to diseases associated with abnormal diruretic, canine natriuretic and vasodilatory activity. Fusion proteins according to the invention may be present in compositions that include pharmaceutically acceptable excipients, carriers or diluents.

The invention relates to fusion proteins as described herein that may have one of the following general formulas, A or B,

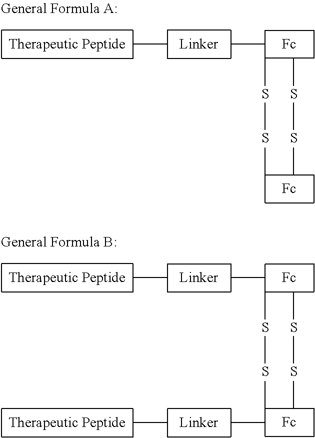

wherein, the therapeutic peptide or protein is

(i) is a non-immunoglobulin polypeptide and

(ii) may be in an orientation of N′ to C of the amino acid sequence, C to N′ of the amino acid sequence or in the case of more than one therapeutic peptide, N′ to C, C to N′ or a mixture of N′ to C and C to N′; and the Fc domain is an IgG heavy chain comprising hinge, CH2 and CH3 regions, wherein the IgG heavy chain begins at the first N-terminal cysteine residue within the hinge region and forms a homodimer with another Fc domain at the first and second N-terminal cysteine residues, and wherein the Fc domain is denoted by FCAB or FCBA, wherein AB is an orientation of N′ to C of both Fc domains and BA is an orientation of C to N′ of both Fc domains. The FC sequences preferably have the sequence of SEQ ID NO:9 and/or SEQ ID NO:10. For example, in either General Formula A or General Formula B, both Fc Domains may have the sequence of SEQ ID NO:9; or both Fc domains may have a sequence of SEQ ID NO: 10, or one of the Fc domains may have a sequence of SEQ ID NO:9 and the other have a sequence of SEQ ID NO:10.

The therapeutic protein in the general formula above may be any therapeutic protein. For example, it may be selected from the group consisting of a cytokine, a ligand-binding protein, a hormone, a neurotrophin, a neutrophin receptor, a body-weight regulator, a serum protein, a clotting factor, a protease, an extracellular matrix component, an angiogenic factor, an anti-angiogenic factor, an immunoglobulin receptor, a blood factor, a cancer antigen, a statin, a growth factor, a therapeutic peptide, a non-human protein, a non-mammalian protein and a protein toxin; in specific examples, the therapeutic peptide or protein is selected from the group consisting of one or more canine ANP, canine BNP, canine urodilatin, canine DNP and a biologically active sequence variant thereof; preferably has the sequence of SEQ ID NO:8; still other exemplary therapeutic proteins include cytokine such as hematopoietic factor, interferon, interleukin and tumor necrosis factor; hematopoietic factors such as erythropoietin, granulocyte-macrophage colony stimulating factor and granulocyte colony stimulating factor; ligand-binding proteins such as CD molecule, CTLA-4, TNF receptor, and interleukin receptor

As contemplated herein, in one aspect this invention embodies fusion proteins comprising at least one canine natriuretic peptide conjugated to the Fc domain of an antibody by way of a linker. The fusion protein may actually comprise one canine natriuretic peptide or two canine natriuretic peptides conjugated to an antibody Fc domain from a canine antibody. As described in detail below, the sequence and length of the linker employed to conjugate the peptide with the Fc domain may vary depending on whether the fusion protein comprises one or two canine natriuretic peptides.

One aspect of the invention relates to a fusion protein having one of the following general formulas, 1 or 2,

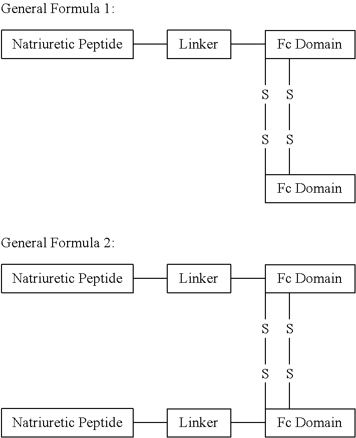

wherein, the natriuretic peptide (i) is selected from the group consisting of one or more canine ANP, canine BNP, canine urodilatin, canine DNP and a biologically active sequence variant thereof and preferably has a sequence of SEQ ID NO: 8 (ii) may be in an orientation of N′ to C of the amino acid sequence, C to N′ of the amino acid sequence or in the case of more than one canine natriuretic peptide, N′ to C, C to N′ or a mixture of N′ to C and C to N′; the linker is one or more linkers selected from the group consisting of a succinate-glycine linker (L1), a GlyGly linker (L2), a Gly(SerGlyGly)2SerGly linker (L3), a (GlyGlySer)_{y}GlyGly linker, wherein y is 3 to 6 (L4), and 7 (L6)) (GlyGlySer)5Gly (L5) and a (GlySerGly)5Gly linker (L5a); and the Fc domain is an IgG heavy chain comprising hinge, CH2 and CH3 regions, wherein the IgG heavy chain begins at the first N-terminal cysteine residue within the hinge region and forms a homodimer with another Fc domain at the first and second N-terminal cysteine residues, and wherein the Fc domain is denoted by FCAB or FCBA, wherein AB is an orientation of N′ to C of both Fc domains and BA is an orientation of C to N′ of both Fc domains. The FC sequences preferably have the sequence of SEQ ID NO:9 and/or SEQ ID NO:10. For Example, in either General Formula 1 or General Formula 2, both Fc Domains may have the sequence of SEQ ID NO:9; or both Fc domains may have a sequence of SEQ ID NO: 10, or one of the Fc domains may have a sequence of SEQ ID NO:9 and the other have a sequence of SEQ ID NO:10.

In another aspect of this invention, the fusion protein has the following formula 3,

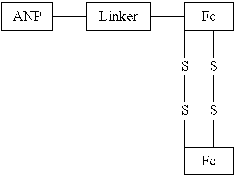

wherein ANP is in an orientation of N′ to C (ANP_{XY}) of the amino acid sequence of ANP or in an orientation of C to N′ (ANP_{yx}) of the amino acid sequence of SEQ ID NO:8; the linker is one or more linkers selected from the group consisting of L1, L2, L3, L4, L5, L5a and L6, wherein L1 is a glycine succinate linker as described herein, L2 is a GlyGly linker, L3 is a Gly(SerGlyGly)2SerGly linker, L4 is a (GlyGlySer)3GlyGly linker, L5 is a (GlyGlySer)5Gly linker, L5a is a (SerGlyGly)5Gly and L6 is a (GlyGlySer)6GlyGly linker; and Fc is (i) an IgG heavy chain comprising hinge, CH2 and CH3 regions, wherein the IgG heavy chain begins at the first N-terminal cysteine residue within the hinge region and forms a homodimer with another Fc at the first and second N-terminal cysteine residues, and (ii) is denoted by FCUB, FCIBA, FC2AB or Fc2BA, wherein Fc1 is derived from an IgG1 molecule, Fc2 is derived from a IgG2 molecule, AB is an orientation of N′ to C of the Fc and BA is an orientation of C to N′ of the Fc. The FC sequences preferably has the sequence of SEQ ID NO:9 and/or SEQ ID NO:10. For Example, in General Formula 3 both Fc domains may have the sequence of SEQ ID NO:9; or both Fc domains may have a sequence of SEQ ID NO: 10, or one of the Fc domains may have a sequence of SEQ ID NO:9 and the other have a sequence of SEQ ID NO:10.

In yet another aspect of this invention, the fusion protein has the following formula 4,

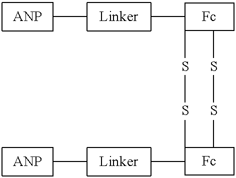

wherein, ANP is in an orientation of N′ to C (ANPXY) of the amino acid sequence of SEQ ID NO:8 or in an orientation of C to N′ (ANPyx) of the amino acid sequence of SEQ ID NO:8; Linker is one or more linkers selected from the group consisting of L1, L2, L3, L4, L5, L5a, and L6 wherein L1 is a glycine succinate linker as described herein, L2 is a GlyGly linker, L3 is a Gly(SerGlyGly)2SerGly linker, L4 is a (GlyGlySer)3GlyGly linker, L5 is a (GlyGlySer)5Gly linker, L5a is a (SerGlyGly)5Gly and L6 is a (GlyGlySer)6Gly Gly linker; and Fc is (i) an IgG heavy chain comprising hinge, CH2 and CH3 regions, wherein the IgG heavy chain begins at the first N-terminal cysteine residue within the hinge region and forms a homodimer with another Fc at the first and second N-terminal cysteine residues, and (ii) is denoted by FCUB, FCIBA, FC2AB or Fc2BA, wherein Fc1 is derived from an IgG1 molecule, Fc2 is derived from a IgG2 molecule, AB is an orientation of N′ to C of the Fc and BA is an orientation of C to N′ of the Fc. Preferably, the FC sequences have the sequence of SEQ ID NO:9 and/or SEQ ID NO:10. For example, in General Formula 4 both Fc domains may have the sequence of SEQ ID NO:9; or both Fc domains may have a sequence of SEQ ID NO: 10, or one of the Fc domains may have a sequence of SEQ ID NO:9 and the other have a sequence of SEQ ID NO:10.

The fusion proteins of the present invention are biologically active molecules, e.g., they are able to catalyze cGMP, but are more useful for therapeutic purposes as they possess much longer half-lives and are also less susceptible to proteolytic degradation. In addition, by exploiting FcRn mediated transport, the therapeutic fusion proteins disclosed herein may be administered by bolus injection but may display pharmacokinetic properties resembling that of a slow-release depot formulation.

The fusion proteins of the present invention are therapeutic peptides that are conjugated to a Fc region of canine antibody, such as a canine IgG directly or through a linker and the Fc region specifically includes a hinge region from a canine IgG. By conjugating the peptide to the Fc region of an antibody, these fusion proteins exhibit much longer half-lives than the unconjugated peptides. Selection of a canine Fc hinge region is important to the in vivo uptake of the therapeutic protein or peptide. Without being limited by any particular mode of action, the fusion proteins of the present invention may be pinocytosed and sequestered upon binding of the Fc region to the neonatal constant region fragment receptor (FcRn) and by exploiting the FcRn active carrier system, (the FcRn pathway transports maternal antibodies (IgG) across the intestinal epithelium of a newborn animal), levels of the fusion proteins disclosed herein can be protected from intracellular lysozomal degredation as well as have reduced exposure to neutral endopeptidase (NEP) or the NPR clearance receptor. The fusion protein may be recycled and represented to the circulation upon normal release from the cell. In this way, activation of the NPRA receptor all at once, such as typical after a bolus dose of ligand, may be avoided. The bioavailability of the fusion proteins of the present invention may more closely resemble a slow-release depot preparation.

The FcRn receptor is expressed on the surface of endothelial cells in several different types of tissue in adult humans, including lung, kidney and intestine. Without being limited by any particular mode of action, the normal function of the FcRn receptor may be exploited as a means to administer bioactive therapeutic-Fc fusion proteins for a myriad of clinical uses. For example, in addition to methods to treat or ameliorate pathological conditions of the cardiovascular system, the fusion proteins of the present invention may be used in methods to treat diseases associated with abnormal diuretic, canine natriuretic and vasodilatory activity in which activation of the NPRA receptor confers a therapeutic benefit on the subject.

In some exemplary embodiments, the fusion proteins of the present invention may comprise any canine natriuretic peptide, including but not limited to canine ANP or canine BNP.

It is also contemplated that in addition to sequence variants, fragments of said peptides are included within the scope of the invention disclosed herein, where such fragments are of sufficient size to be therapeutically effective in the methods of the present invention. One of skill in the art can determine alterations in peptide length and sequence variety that does not impact biological activity and/or therapeutic effectiveness without undue experimentation. The proteins may be in the form of acidic or basic salts, or may be in a neutral form. Individual amino acid residues may also be modified by oxidation or reduction.

Other variants within the scope of the invention include fusion proteins in which the primary amino acid structure is modified by forming covalent or aggregative conjugates with other peptides or polypeptides, or chemical moieties such as glycosyl groups, lipids, phosphate, acetyl groups and the like. Covalent derivatives may be prepared, for example, by linking particular functional groups to amino acid side chains or at the N- or C-terminus.

The fusion proteins of the present invention may or may not be glycosylated. Fusion proteins expressed in yeast or mammalian expression systems may be similar to, or slightly different in molecular weight and glycosylation pattern from the native molecules, depending upon the expression system; expression of DNA encoding polypeptides in bacteria such as E. coli provides non-glycosylated molecules.

In certain embodiments, dimer constructs of the present invention have been found to possess increased in vitro potency when compared to monomer constructs. The increased potency of dimer constructs of therapeutic peptides linked to canine antibody Fc domains as described herein is particularly surprising in view of the monomeric interaction between the canine natriuretic peptide ligands (e.g., ANP, BNP, etc.) and their cell surface receptors. It would be expected that dimeric constructs as described herein would be sterically hindered from interacting with the cells and/or receptors and thus, little and/or no activity of such dimeric constructs would be predicted.

In certain embodiments, monomer constructs of the present invention have been found to possess increased in vivo serum concentrations (Cmax) when compared to dimer constructs. The increased Cmax of monomer constructs of canine natriuretic peptides linked to antibody Fc domains as described herein is surprising in view of the previous results from intravenous administration of monomeric EPO-Fc constructs that showed a lower Cmax as compared to intravenous administration of dimeric EPO-Fc constructs (see, e.g., Table 4 of U.S. Patent Application Publication No. 2007/0172928).

The canine Fc domain conjugated to the therapeutic peptide(s) is preferably the Fc domain of canine IgG, including, but not limited to, IgGA, IgGB, IgGC or IgGD. However, Fc domains of other antibodies may be used if modified to possess minimal or no effector function. Canine antibody Fc domains are preferred, but other species types, wild-type forms as well as sequence variants, may be used, e.g., a recombinant Fc molecule is described in the Examples provided herein. In one aspect of this invention, the Fc domain is made up of two Fc heavy chains from IgG1 or IgG2 isotypes with the hinge residues removed down to the CPPCP sequence on each chain to allow for interchain disulfide bonding of the cysteine residues. Ideally, the fusion proteins of the present invention comprise a canine Fc domain that is able to bind to the FcRn receptor, trigger the active carrier function of this receptor and cause delivery of the fusion protein into the cell without causing an adverse ADCC toxic response. Once inside the cell, pH changes result in the release of the fusion protein from the FcRn receptor, and the fusion protein may ultimately be released from the cell back into the circulation. (Roopenian D. C. et. al. (2003) J. Immunology 170:3528-3533; Lencer, W. I. et al, Trends in Cell Biology 15(1):5-9 (2005)).

The length and sequence of the amino acid linkers used to conjugate the therapeutic peptide with the canine Fc domain may be varied. Structural modeling of NPRA with canine ANP bound in relation to a canine Fc domain can be used to predict the minimum linker distance required to allow insertion of the canine Fc-fused canine ANP into the NPRA active site. The linker length should be one that minimizes steric and electrostatic repulsions between the canine natriuretic peptide and the Fc domain. For example, the desired minimal distance from the C-terminus of ANP is 12 A from the closest N-terminus of the Fc homodimer and 17 A from the other N-terminus of the Fc homodimer. Furthermore, if the Fc homodimer has only one ANP fused, (e.g., monomer) a 4 to 6 amino acid minimum linker length would suffice. With two ANP peptides bound to the Fc homodimer (e.g., dimer), and with only one ANP bound to NPRA receptor (i.e., in a 1 :1 Fc dimer :NPRA ratio) then a longer linker length e.g. of 9 amino acids for each linker may be preferred. For both ANPs to bind to NPRA (e.g., to two adjacent receptors or to 1 NPRA receptor in nonstatic, alternating fashion) then linkers with an exemplary length of 12 amino acids could be used. Increasing linker length may have beneficial effects on the properties of the fusion proteins. For example, increased linker length may allow the fusion proteins to approach the potency of the fused canine natriuretic peptide. For example, longer linker lengths (e.g., 20 amino acids in length) may increase potency of the canine natriuretic peptide (as measured by ability to induce cGMP in vitro.

Thus, where increasing linker length increases potency of the fusion proteins. In embodiments of this invention, the potency of the fusion protein may thus be improved at a linker length of between 16 and 50 amino acids, preferably between 16 and 40 amino acids and most preferably between 16 and 30 amino acids.

Linker sequences employed in the present invention comprising (GGS)x repeats (e.g., where x is an integer from 0 to 16), may be made according to conventional synthetic, semi-synthetic, or recombinant methods (see, e.g., Evers T. H. et. al. (2006) Biochemistry, 45:13183-13192). With regard to the actual amino acid sequence of the linkers employed, typically glycines and serines are preferred, as the presence of glycines in the linker provide flexibility and serines provide solubility. A preferred linker sequence is made up of a series of repeats of these amino acids, e.g., (GGS)x-GG, for example, where x is an integer from 0 to 16, such as GGSGGSGGSGG or GGSGGSGGSGGSGGSGGSGG. This latter sequence of GGSGGSGGSGGSGGSGGSGG is particularly preferred.

As mentioned above, the orientation of conjugation of Fc domain and therapeuitc peptide may vary. For example, the carboxy terminus of the therapeutic peptide may be linked to the amino terminus of the canine Fc domain by a normal peptide bond. Alternatively, the amino terminus of the therapeutic peptide may be linked to the amino terminus of the canine Fc domain. In the latter case, the chemistry leaves a succinate moiety in place of one amino acid of the fusion. As such, one of skill in the art will recognize that fusion proteins of the latter case may not be made recombinantly as normal peptide bonding does not take place between two amino termini. Data gathered indicate that orientation does not seem to effect the potency of a given fusion protein.

Specific therapeutic constructs of the invention are exemplified using canine ANP fused to a canine Fc domain wherein one or more canine ANP, one or more linkers and one or more canine Fc are used. The following canine ANP-canine Fc fusion constructs are contemplated by the present invention.

An exemplary fusion construct (Construct 2), which comprises ANPXY (Construct 1), is represented by:

ANP_{XY}-L4-Fc1ab

The construct comprises ANPXY-L4-FC1AB, wherein ANPXY has a sequence of SEQ ID NO: 8, L is GGSGGSGGSGGSGGSGGSGG SEQ ID NO: 12 and FC1ab has a sequence of SEQ ID NO: 9. ANPXY-L-FC1AB is represented by SEQ ID NO: 3. When the ANPXY-L-Fc1ab construct is made recombinantly, a homodimer may be produced, for example, the ANPXY-L-FC1ab may be linked to a second ANPXY-L-FC1ab construct via a disulfide linkage.

An exemplary fusion construct (Construct 3) is represented by:

ANPXY-L-FC2ab

wherein ANPXY has a sequence of SEQ ID NO: 8, L is GGSGGSGGSGGSGGSGGSGG SEQ ID NO: 12 and FC2ab has a sequence of SEQ ID NO: 10. ANPXY-L-FC2ab is represented by SEQ ID NO: 6. When the ANPXY-L-FC2ab construct is made recombinantly, a homodimer may be produced, for example, the ANPXY-L-FC2ab may be linked to a second ANPXY-L-FC2ab construct via a disulfide linkage.

Another exemplary fusion construct (Construct 4) is represented by:

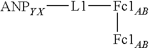

In this construct, the ANP YX is SEQ ID NO: 8 inverted in orientation from its C to N′ terminus, L1 is linker having a sequence of GGSGGSGGSGGSGGSGGSGG (SEQ ID NO:12) and FC1AB is SEQ ID NO: 9 or SEQ ID NO:10. The ANPYX-L1-FC1AB may be linked to a second FC1AB via a disulfide linkage and the second FC1AB may have the same sequence as the first FC1AB or may be different to the sequence of the first FC1AB

Another exemplary fusion construct (Construct 5) is represented by:

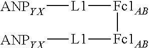

In this construct, ANP YX is SEQ ID NO: 8 inverted in orientation from its C to N′ terminus, L1 is SEQ ID NO:12 and FC1AB is SEQ ID NO: 9 or SEQ ID NO:10. The ANPYX-L1-FC1AB may be linked to a second ANP_{YX}-L1-FC1AB construct via a disulfide linkage and FC1AB in both units may be SEQ ID NO:9, or may be SEQ ID NO:10, or may be SEQ ID NO:9 in one unit and SEQ ID NO:10 in the other.

### C. SYNTHESIS OF THE FUSION PROTEINS

Unless otherwise indicated herein, the fusion proteins of the present invention may be made by any of a number of techniques of protein chemistry or molecular biology familiar to one of skill in the art. (See, e.g., Dawson et al., Ann. Rev. Biochem., 69:923-960, 2000.) Possible synthesis scenarios are described in the Examples provided herein and include synthetic and semi-synthetic chemical synthesis as well as recombinant methods.

Fusion proteins may be produced using chemical methods in whole or in part and using classical or nonclassical amino acids or chemical amino acid analogs as appropriate. Techniques include solid phase chemistry (Merrifield, J. Am. Chem. Soc, 85:2149, 1964; Houghten, Proc. Natl. Acad. Sci. USA 82:5132, 1985) and equipment for such automated synthesis of polypeptides is commercially available (e.g., Applied Biosystems, Foster City, Calif). Synthesized peptides can be purified using conventional methods such as high performance liquid chromatography. The composition of the synthetic fusion polypeptides may be confirmed by amino acid analysis or sequencing using techniques known to one of skill in the art. Further treatment of a synthesized protein under oxidizing conditions may also be utilized to obtain the proper native conformation. See, e.g. Kelley, R. F. & Winkler, M. E. in Genetic Engineering Principles and Methods, Setlow, J. K., ed., Plenum Press, N.Y., vol. 12, pp 1-19, 1990; Stewart, J. M. & Young, J. D. Solid Phase Peptide Synthesis Pierce Chemical Co., Rockford, 111,1984).

The fusion proteins disclosed herein may also be made by recombinant techniques involving gene synthesis, cloning and expression methodologies. These techniques are well known and are explained in, for example, Current Protocols in Molecular Biology, Volumes I, II, and III, 1997 (F. M. Ausubel ed.); Sambrook et al, 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol. 154 and Vol. 155 (Wu and Grossman, and Wu, eds., respectively).

Briefly, the fusion proteins of the present invention may be made recombinantly by isolating or synthesizing nucleic acid sequences encoding any of the amino acid sequences described herein by conventional cloning or chemical synthesis methods. For example, DNA fragments coding for the different fusion protein sequences may be ligated together in-frame in accordance with conventional techniques or synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence. The recombinant nucleic acids can further comprise other nucleotide sequences such as sequences that encode affinity tags to facilitate protein purification protocol.

The nucleic acid sequence encoding a fusion protein of the present invention may be ligated into a suitable expression vector capable of expressing the nucleic acid sequence in a suitable host, followed by transforming the host with the expression vector into which the nucleic acid sequence has been ligated, culturing the host under conditions suitable for expression of the nucleic acid sequence, whereby the protein encoded by the selected nucleic acid sequence is expressed by the host and purifying the protein produced. In this process, the ligating step may further contemplate ligating the nucleic acid into a suitable expression vector such that the nucleic acid is operably linked to a suitable secretory signal, whereby the amino acid sequence is secreted by the host. Suitable secretory signals for use with the present invention include but are not limited to, the mouse IgG kappa light chain signal sequence (Ho et. al. PNAS (2006) 103(25): 9637-9642).

As described above, a nucleic acid sequence encoding a fusion protein described herein may be inserted into an appropriate plasmid or expression vector that may be used to transform a host cell. In general, plasmid vectors containing replication and control sequences that are derived from species compatible with the host cell are used in connection with those hosts. The vector ordinarily carries a replication site, as well as sequences which encode proteins that are capable of providing phenotypic selection in transformed cells. For example, E. coli may be transformed using pBR322, a plasmid derived from an E. coli species (Mandel, M. et al., J. Mol. Biol. 53:154, 1970). Plasmid pBR322 contains genes for ampicillin and tetracycline resistance, and thus provides easy means for selection. Other vectors include different features such as different promoters, which are often important in expression. The vectors used for mammalian expression often contain the constitutive CMV promoter that leads to high recombinant protein expression. These vectors also contain selection sequence genes that are used for the generation of stable expressing cell lines.

Host cells may be prokaryotic or eukaryotic. Prokaryotes are preferred for cloning and expressing DNA sequences to produce parent polypeptides, segment substituted polypeptides, residue-substituted polypeptides and polypeptide variants. Such prokaryotic cells familiar to one skilled in the art include, but are not limited to, E. coli, B subtillus, and P. aeruginosa cell strains. In addition to prokaryotes, eukaryotic organisms, such as yeast cultures, or cells derived from multicellular organisms may be used. Vertebrate cells may also be used as useful host cell lines. Useful cells and cell lines are familiar to one of skill in the art and include, but are not limited to, HEK293 cells, HeLa cells, Chinese Hamster Ovary (CHO) cell lines, WI 38, 293, BHK, COS-7 and MDCK cell lines.

The invention also relates to isolated or purified polynucleotides that encode the canine natriuretic fusion proteins of the present invention. As discussed above, the polynucleotides of the invention which encode a fusion protein, fragments thereof, or functional equivalents thereof may be used to generate recombinant nucleic acid molecules that direct the expression of the fusion protein, fragments thereof, or functional equivalents thereof, in appropriate host cells. The fusion polypeptide products encoded by such polynucleotides may be altered by molecular manipulation of the coding sequence.

Due to the inherent degeneracy of the genetic code, other DNA sequences which encode substantially the same or a functionally equivalent amino acid sequence, may be used in the practice of the invention for the expression of the fusion polypeptides. Such DNA sequences include those which are capable of hybridizing to the coding sequences or their complements disclosed herein under low, moderate or high stringency conditions described herein.

Altered nucleotide sequences which may be used in accordance with the invention include deletions, additions or substitutions of different nucleotide residues resulting in a sequence that encodes the same or a functionally equivalent gene product. The gene product itself may contain deletions, additions or substitutions of amino acid residues, which result in a silent change.

The nucleotide sequences of the invention may be engineered in order to alter the fusion protein coding sequence for a variety of ends, including but not limited to, alterations which modify processing and expression of the gene product. For example, mutations may be introduced using techniques which are well known in the art, e.g., to insert or delete restriction sites, to alter glycosylation patterns, phosphorylation, to create and/or destroy translation, initiation, and/or termination sequences, or to create variations in coding regions, to facilitate further in vitro modification, etc. One of skill will recognize many ways of generating alterations in a given nucleic acid construct. Such well-known methods include, e.g., site-directed mutagenesis, PCR amplification using degenerate oligonucleotides, exposure of cells containing the nucleic acid to chemical mutagenic agents or radiation, chemical synthesis of a desired oligonucleotide (e.g., in conjunction with ligation and/or cloning to generate large nucleic acids) and other well-known techniques.

Purified fusion proteins may be prepared by culturing suitable host/vector systems to express the recombinant translation products of the DNAs of the present invention, which are then purified from culture media or cell extracts. For example, supernatants from systems which secrete recombinant polypeptide into culture media may be first concentrated using a commercially available protein concentration filter, such as, e.g., an Amicon or Millipore Pellicon ultrafiltration unit. Following the concentration step, the concentrate may be applied to a suitable purification matrix. Affinity chromatography or reverse-phase high performance liquid chromatography (RP-HPLC) may also be used to purify the fusion proteins of the present invention.

### D. FORMULATIONS

The therapeutic fusion proteins of the present invention can be administered as veterinary compositions for treatment of specific diseases in animals or can be used to test dosing and efficacy of compounds in dogs as animal models. For example the compositions are for use in methods to treat or ameliorate pathological conditions in which activation of the NPRA receptor confers a therapeutic benefit on the subject, including but not limited to diseases associated with abnormal diruretic, canine natriuretic and vasodilatory activity and/or in which it is desirable to induce naturesis, diuresis, vasodilation or to modulate the renin-angiotensis II and aldosterone systems. Such pathological conditions include disorders of the cardiovascular system such as described in detail above. These conditions include those that may be characterized by an excess in extracellular fluid, including but not limited to Chronic Heart Failure (CHF) and pulmonary edema. In a particularly preferred embodiment, the invention includes methods to treat or ameliorate pathological conditions of the cardiovascular system including but not limited to, chronic heart failure (non-ischemic), post-MI heart failure (ischemic CHF), acute MI, reperfusion injury, left ventricular dysfunction (LVD), cardiac fibrosis, diastolic heart failure, and hypertrophic cardiomyopathy. In addition, hypertensive disorders including, but not limited to hypertension, e.g., pulmonary hypertension, systolic hypertension, resistant hypertension and other cardiovascular related diseases such as diabetic nephropathy may be treated or ameliorated by the methods of the present invention. It is also contemplated herein that the fusion proteins and pharmaceutical compositions of the present invention may be used to provide therapeutic benefit for subjects undergoing coronary artery bypass graft procedures.

Pharmaceutical compositions for use in accordance with the present invention may be formulated in a conventional manner using one or more physiologically acceptable carriers or excipients for administration by various means, for example, by inhalation or insufflation (either through the mouth or the nose) or topical, oral, buccal, parenteral or rectal administration. For example, parenteral may include, intradermal, intramuscular, intraperitoneal, intravenous, subcutaneous, intranasal and epidural administration. The compounds may be administered by any convenient route, for example by infusion or bolus injection, by absorption through epithelial or mucocutaneous linings (e.g. oral mucosa, rectal and intestinal mucosa, etc.) and may be administered together with other biologically active agents. Administration can be systemic or local. In a preferred embodiment, it may be desirable to introduce the pharmaceutical compositions of the invention into the affected tissues by any suitable route. Pulmonary administration can also be employed, e.g., by use of an inhaler or nebulizer, and formulation with an aerosolizing agent.

The pharmaceutical compositions may further comprise a vehicle or carrier, including a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the pharmaceutical composition is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin, which is herein incorporated by reference in its entirety. The formulation should suit the mode of administration.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Where appropriate, preparations for oral administration may be suitably formulated to give controlled release of the active compound.

Where appropriate, for buccal administration the compositions may take the form of tablets or lozenges formulated in conventional manner.

In another embodiment, the fusion proteins of the present invention are for administration by inhalation or insufflation (either through the mouth or the nose). As such, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

In a particularly preferred embodiment, the pharmaceutical compositions of the present invention may be formulated for parenteral administration by injection, e.g., by bolus injection or continuous infusion. In addition, as contemplated herein, the fusion proteins and pharmaceutical compositions of the present invention may be suitable for self-injection by a subject in need thereof, e.g. long term treatment of CHF. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Alternatively, the active ingredient may be in powder form for constitution with a suitable vehicle, e.g., sterile pyrogen-free water, before use. For example, lyophilized protein compositions may be inhaled or reconstituted then injected in a suitable vehicle.

The compounds may also be formulated in rectal compositions such as suppositories or retention enemas, e.g., containing conventional suppository bases such as cocoa butter or other glycerides.

In addition to the formulations described previously, which may exhibit pharmacokinetics similar to a slow release formulation, the compounds may also be formulated as an actual depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

Pharmaceutical compositions suitable for use in the invention include compositions wherein the active ingredients are contained in an effective amount to achieve the intended purpose. The determination of an effective dose is well within the capability of those skilled in the art. For example, for any compound, the therapeutically effective dose can be estimated initially either in cell culture assays, e.g., of neoplastic cells, or in animal models, usually mice, rabbits, dogs, or pigs. The animal model may also be used to determine the appropriate concentration range and route of administration. A dose may be formulated in animal models to achieve a circulating plasma concentration range that includes the IC50 (i.e., the concentration of the test compound that achieves a half-maximal inhibition of symptoms). Such information can then be used to determine useful doses and routes for administration in humans.

A therapeutically effective dose or “effective amount” refers to that amount of active ingredient that is nontoxic but sufficient to provide the desired therapeutic effect. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage form employed, sensitivity of the subject, and the route of administration.

The exact dosage will be determined by the practitioner, in light of factors related to the subject that requires treatment. Dosage and administration are adjusted to provide sufficient levels of the active moiety or to maintain the desired effect. Factors that may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc. Pharmaceutical formulations suitable for oral administration of proteins are described, e.g., in U.S. Pat. Nos. 5,008,114; 5,505,962; 5,641,515; 5,681,811; 5,700,486; 5,766,633; 5,792,451; 5,853,748; 5,972,387; 5,976,569; and 6,051,561.

The present invention further provides kits for use with any of the above methods. Such kits typically comprise two or more components necessary for performing a method described herein. Components may be compounds, reagents, containers and/or equipment. For example, one container within a kit may contain a pharmaceutical composition comprising fusion proteins of the present invention. One or more additional containers may enclose elements, such as reagents or buffers, or equipment to be used in a method to administer the pharmaceutical composition.

It is also contemplated herein that the fusion proteins and pharmaceutical compositions of the present invention may be administered alone or in combination with other compounds or substances that may be used to treat any of the pathological conditions described herein. Such compounds or formulations that may be used in combination therapy with the present invention include, for example, diuretics, beta blockers, and Ang II receptor blockers.

It is contemplated that the invention described herein is not limited to the particular methodology, protocols, and reagents described as these may vary. It is also to be understood that the terminology used herein is for the purpose of describing particular embodiments only, and is not intended to limit the scope of the present invention in any way.

Although any methods and materials similar or equivalent to those described herein can be used in the practice or testing of the present invention, the preferred methods, devices and materials are now described. All references described herein are expressly incorporated by reference.

### E. EXAMPLES

### Example 1

### Recombinant Canine ANP-Fc Fusion Protein Production and Characterization

Canine ANP-Fc Fusion Construct Generation: Two DNA constructs (SEQ ID 1 and 4) are synthesized according to conventional oligonucleotide synthesis techniques, and subcloned into the mammalian expression vector pcDNA3.1 (Geneart). Each construct contains the same basic order of a mouse IgG kappa light chain signal sequence, canine ANP28 sequence, a linker sequence followed by a canine Fc gamma sequence. The mouse IgG kappa light chain signal sequence utilized is METDTLLLWVLLLWVPGSTG (accession number AAA38778-SEQ ID NO7). The canine ANP28 sequence is from the NCBI database with the accession number P07499 (SEQ ID NO8). The linker sequence encodes for a 20 aa peptide consisting of repeats of glycine and serine residues in a (GGS)₆GG motif. In one construct the linker is attached to the Fc region of canine IgG-A (accession number AAL3530) while in the second construct the linker is attached to the Fc region of canine IgG-B (accession number AAL35302). The Fc regions of canine IgG-A and IgG-B isotypes [Vet Immunol and Immunopath (2001) 80(3-4): 259-270] are utilized due to their predominance in canines and their similarity in hinge structure to human IgG1 and IgG2. The Fc fusions to both IgG-A and IgG-B isotypes are generated in such a way that the hinge region is chopped down to include only two cysteine residues (CTDTPPCP (SEQ ID NO:18) for IgG-A Fc and CPKCP (SEQ ID NO:19) for IgG-B Fc) ensuring that the disulfide linkage in the hinge would be similar on the two Fc isotypes. Each construct was codon optimized for mammalian expression using standard codon tables.

Mammalian Expression of the Canine ANP-Fc Fusions: The initial expression of all the canine ANP-Fc fusion constructs is done from HEK293 cell lines using a transient transfection protocol at the 1L scale. HEK293 cells are grown in FreeStyle 293 medium and transfected using the 293Fectin reagents and protocols as described by the manufacturer (Invitrogen). Transiently transfected cells are grown in a spinner flask with agitation of 90 rpm at 37° C. and 5% CO₂. The cells are monitored daily for cell density, viability, and diameter, level of glucose, lactate, glutamine and pH. The conditioned media is harvested 72-96 hours after the transfection by centrifugation at 3600 rpm for 15 minutes and is delivered fresh to purification. Expression levels are determined by SDS-PAGE and Western-blotting analysis. Once the expression levels are determined for each construct, stable pools are generated, if needed, to produce the protein required. The stable pools generation started in a flask where stable cells are selected with 400 μg/ml of G418 and then transferred into spinner. Once the cells stabilize under these conditions, cells are expanded and 5-25L runs are carried out in wave bioreactors.

Purification of the Recombinant Canine ANP-Fc Fusion Proteins: All the purifications are performed at 4° C. using an AKTA system (GE Healthcare). Five milliliters of Protease Inhibitor Cocktail (Sigma) are added per liter of conditioned media. A column is packed with 3-4 mL ProteinA Ceramic HyperD®F resin (Pall) per liter of conditioned media and equilibrated with 10 CV of Equilibration Buffer (1× Dulbecco's PBS without Mg/Ca, pH 7.3, Invitrogen) at 1 mL/min. Conditioned media is loaded onto the column overnight at 1-5 mL/min depending upon the volume of media. The resin is then washed, in 10 CV sequences, with Wash Buffer 1 (DPBS without Mg/Ca, pH 7.3), Wash Buffer 2 (DPBS without Mg/Ca pH 7.3+1 M NaCl), and then Wash Buffer 1 again. The bound protein is eluted with 10 CV Elution Buffer (0.1 M Glycine/HCl, pH 2.5 in dH₂O) at 2.5 ml/min. The 3 mL fractions are neutralized immediately with 300 μL (10% of fraction) of Neutralization Buffer (1 M Tris-HCl pH 8.0, Invitrogen). Fractions containing canine ANP-Fc fusion protein are pooled and loaded onto a column packed with Q Sepharose (GE Healthcare) anion exchange resin linked in series to column packed with S Sepharose (GE Healthcare) cation exchange resin (both columns were pre-equilibrated with 20 mM Sodium Citrate, pH 6.0). After loading the columns were washed with 10 CV of 20 mM Sodium Citrate, pH 6.0. The Q column was then disconnected from the system and the S column was eluted with a gradient up to 1 M NaCl. The canine ANP-Fc fusion containing fractions were concentrated using Amicon Ultra-15 10 kDa MWCO concentrators (Millipore) to approximately 3 mg/mL. Aliquots are flash-frozen in liquid nitrogen and then stored at −80° C. Final products are characterized by SDS-PAGE (non-reduced and reduced), analytical ultracentrifugation and mass spectrometric analysis before submitted for biological function assays. Endotoxin contaminant levels are determined using the EndoSafe PTS System (Charles River Labs).

The canine ANP-Fc fusion constructs are initially produced using 1L transient mammalian expression. The expressed product is purified through a three step process (Protein A, Q anion exchange, S cation exchange) to generate high quality protein. This transient production process yields only ˜1 mg/L of ANP-Fc(CaIgG-A) fusion and 25-30 mg/L ANP-Fc(CaIgG-B) fusion that is >90% pure (SDS-PAGE). To meet the increasing protein demands stable pools were generated and large-scale wave bioreactor productions were implemented. These stable pools enabled the generation of gram quantities of the canine ANP-Fc fusions.

Analytical Ultracentrifugation of the Recombinant Canine ANP-Fc Fusion Proteins: Sedimentation velocity experiments are conducted to assess the purity and aggregate content of the purified canine ANP-Fc fusions. The canine ANP-Fc fusions are evaluated by sedimentation velocity in a phosphate buffered saline buffer containing 10 mM Sodium Phosphate, 150 mM NaCl, pH 7.3. Samples are loaded into centrifuge cells containing double-sector charcoal-epon centerpieces and quartz windows. Data is collected using a Beckman XLI analytical ultracentrifuge at 280 nm, 50,000 rpm and 20° C. Solution densities and viscosities are measured using an automated Anton-Paar AMVn/SP3-V viscometer and DMA4500/DMA5000 densitometer at 20° C. The sedimentation data are analyzed using the program SEDFIT (v9.3b).

Mass Spectroscopy Analysis of the Recombinant Canine ANP-Fc Fusions: Mass spectrometric experiments are conducted to measure intact molecular weight, to perform partial sequence validation via enzymatic digestion and peptide mapping, and to identify degradation products of the canine ANP-Fc fusions.

To evaluate intact molecular weight 2 μl of fusion protein is injected onto an Agilent Poroshell column (5 um, 1.0×75 mm) at a flow rate of 100 μl/min. The protein is eluted with a gradient from 20% to 65%A over 6 minutes [Mobile phase A is 98/2/0.1 (Water/Acetonitrile/Formic Acid) and Mobile phase B is 95/5/0.1 (Acetonitrile/Water/Formic Acid)]. The effluent is flowed into an Agilent 6210 LC/TOF mass spectrometer scanning from 600-2500 amu with a 2 second scan time. Data is deconvoluted using the Agilent TOF Protein Confirmation software to yield molecular weight(s).

The fusion proteins are digested with trypsin or Endo-LysC (8010-108) for peptide mapping and degradation analysis. Briefly, 25 μl of each sample is denatured, reduced, and alkylated. Trypsin or Endo-LysC is added, and the sample incubated overnight in a 37° C. water bath. Analysis is performed via HPLC/FTMS. The instrument generates multiple scans during the chromatographic separation at approximately 50,000 resolution. The mass of each eluting peptide is determined with an accuracy of 0.0005% relative error. Peptides predicted based on enzyme specificity are matched if the theoretical value is within 0.0005% of the experimentally determined value. The Endo-LysC data is also searched for N-terminal truncations by looking for the masses corresponding to possible amino acid deletions. Unlike trypsin, the Endo-LysC experiment allows for observation of the intact amino terminal region.

The above-described characterization platform was implemented to ensure the quality of the recombinantly produced canine ANP-Fc fusions being tested. As canine ANP28 is very potent in the canine NPRA cGMP assay (EC50 ˜1 nM) monitoring the amounts of “free” ANP in all of the ANP-Fc fusion lots generated using mass spectrometric tools (intact mass and tryptic digests) was critical. These mass spectrometric analyses also allowed us to ensure that the many critical aa's and disulfide, that are necessary for functionality in the canine ANP28 structure, were intact. While the major glycosylation structures (G0, G1, and G2) were identified in the intact mass analysis, a complete glycan analysis was not performed. A mass spectrometric Endo-LysC digest protocol was also implemented to evaluate the N-terminal end of the ANP-Fc fusion peptides. Lots with >90% intact N-termini were selected for further analysis. All lots produced were found to be missing their C-terminal Lysine residue. The level of aggregation was tested by sedimentation velocity experiments in an analytical ultracentrifuge where all lots were found to contain >85% monomer.

### Example 2

### Generation of Stable Cell Lines for In Vitro NPRA Cell Assays

Full-length NPRA sequence containing plasmids are purchased from OriGene Technologies, Inc. (Rockville, Md.) then subcloned into pcDNA3.1. Insert orientation and nucleotide sequence of each construct is verified by an outside vendor (SeqWright, Inc.). The pcDNA3.1-NPRA clones are transfected using Lipofectamine (Invitrogen) into HEK293 cells where stable cell lines expressing NPRA are selected using G418. Clones are screened using the natriuretic peptide induced cGMP assay described below [NPRA clones are treated with ANP (Sigma)]. High cGMP producing clones are expanded. Cell lines are grown in DMEM containing, 100 μg/ml penicillin/streptomycin, L-glutamine, 400 μg/ml of G418, and 10% FBS (Hyclone).

### Example 3

### Natriuretic Peptide Fusion Protein Induced cGMP Assay

Natriuretic peptides of >95% HPLC purity are obtained from Sigma. HEK293 NPRA cells grown to 90% confluence are harvested using HANKS based Cell Dissociation Medium (GIBCO). Cells are washed and resuspended at 3.3×10⁵ cells per mL in pre warmed Dulbecco's PBS, pH 7.4, 25 mM HEPES, 0.1% BSA, 500 μM 3-Isobutyl-1-methylxanthine (IBMX) [Assay Buffer]. Assays are performed in Optiplate-96 White Opaque 96-well Microplates (Perkin Elmer). 15 μL of cell suspension is added to 15 μL of 2× fusion protein in Assay Buffer in triplicate and incubated for 20 minutes at 37° C. The cGMP concentration is measured using the HitHunter™ cGMP Assay Kit (DiscoveRx Corporation). cGMP production dose response curves are generated with a four parameter logistic equation fitted using the Levenburg Marquardt algorithm in XLfit4.2 data analysis software (ID Business Solutions, Ltd.).

### Example 4

### Recombinant Canine ANP-Fc Fusion Proteins

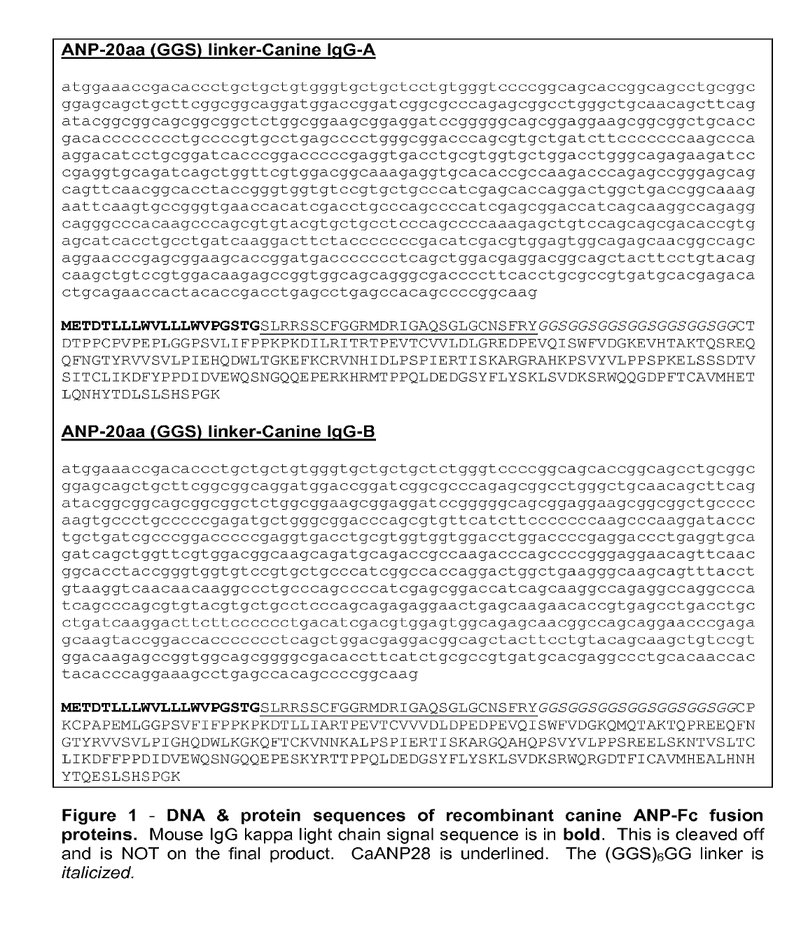

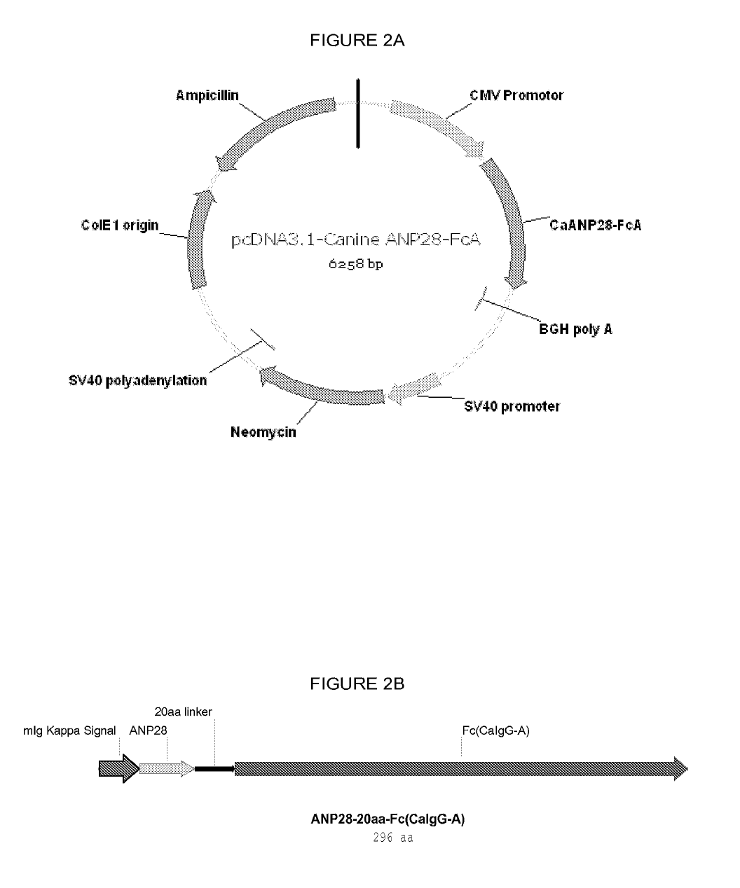

To produce recombinant canine ANP-Fc fusion proteins mammalian expression constructs are generated. These fusion constructs are designed with common signal sequence, canine ANP28 sequence, linker sequence, and either the Fc region of canine IgG-A or IgG-B. These fusion proteins each contain a N-terminal mouse IgG kappa light chain signal sequence (SEQ ID NO7) that is cleaved off and not part of the final protein product. A 20 aa Glycine-Serine linker is utilized as Glycines add flexibility, while Serines add solubility. The Fc regions of canine IgG-A and IgG-B isotypes [Vet Immunol and Immunopath (2001) 80(3-4): 259-270] are utilized due to their predominance in canines and their similarity in hinge structure to human IgG1 and IgG2. The Fc fusions to both IgG-A and IgG-B isotypes are generated in such a way that the hinge region is chopped down to include only two cysteine residues (CTDTPPCP (SEQ ID NO:18) for IgG-A Fc and CPKCP (SEQ ID NO:19) for IgG-B Fc) ensuring that the disulfide linkage in the hinge would be similar on the two Fc isotypes. The respective DNA and protein sequences (SEQ ID NO:1-6) of the recombinant canine ANP-Fc fusions generated are detailed in FIG. 1. Expression of the canine fusion protein constructs is driven by the strong CMV promoter of the pcDNA3.1 mammalian expression vector (FIG. 2).

A production platform was developed for the generation of recombinant canine ANP-Fc fusions. The canine ANP-Fc fusion constructs are initially produced using 1L transient mammalian expression. The expressed product is purified through a three step process (Protein A, Q anion exchange, S cation exchange) to generate high quality protein. This transient production process yields only ˜1 mg/L of ANP-Fc(CalgG-A) fusion and 25-30 mg/L ANP-Fc(CalgG-B) fusion that is >90% pure (SDS-PAGE). To meet the increasing protein demands stable pools were generated and large-scale wave bioreactor productions were implemented. These stable pools enabled the generation of gram quantities of the canine ANP-Fc fusions. The predicted MW of the non-reduced CaANP-Fc fusion is 60.4 kDa and 59.4 kDa (IgG-A and IgG-B Fc fusion respectively), while the predicted MW of the reduced CaANP-Fc fusion is 30.2 kDa and 29.7 kDa (IgG-A and IgG-B Fc fusion respectively).

A characterization platform was implemented to ensure the quality of the recombinantly produced ANP-Fc fusions being tested. As canine ANP28 is very potent in the canine NPRA cGMP assay (EC50 ˜1 nM) monitoring the amounts of “free” ANP in all of the ANP-Fc fusion lots generated using mass spectrometric tools (intact mass and tryptic digests) was critical. These mass spectrometric analyses also allowed us to ensure that the many critical aa's and disulfide, that are necessary for functionality in the canine ANP28 structure, were intact. While the major glycosylation structures (G0, G1, and G2) were identified in the intact mass analysis, a complete glycan analysis was not performed. A mass spectrometric Endo-LysC digest protocol was also implemented to evaluate the N-terminal end of the ANP-Fc fusion peptides. Lots with >90% intact N-termini were selected for further analysis. All lots produced were found to be missing their C-terminal Lysine residue. The level of aggregation was tested by sedimentation velocity experiments in an analytical ultracentrifuge where all lots were found to contain >85% monomer.

Overall these processes allowed for the production of grams of recombinant protein for in vitro and in vivo analysis. Also, the final protein from these runs was found to have low amounts of aggregation (<10%), low endotoxin (<1 EU/ml) and to be intact.

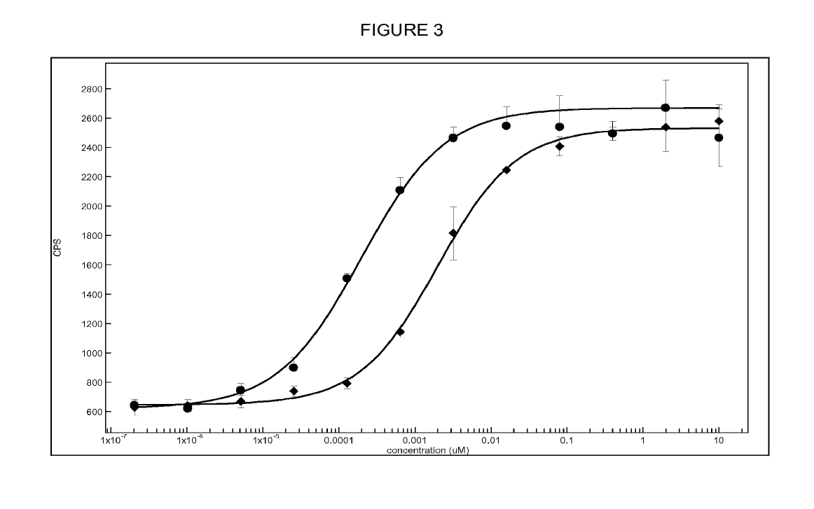

The recombinantly produced canine ANP-Fc fusion constructs are tested in canine NPRA cGMP induction assays (Table 1). A representative cGMP assay data set is graphed in FIG. 3. The shifts in potency seen with the recombinant canine ANP-Fc fusion proteins are visualized by a shift in the curve to the right. The canine ANP-Fc fusions have potencies that are shifted anywhere from 10 to 20-fold from ANP28 depending on the lot tested.

TABLE 1
Recombinant Canine ANP-Fc fusions and cGMP
Induction Response Generated on
Canine NPRA Cells (ANP EC50 = 0.2 +/− 0.07 nM)

| SEQ | | | |
|---|---|---|---|
| ID | | | cGMP EC50 (nM) |
| NO. | Construct | Lot No. | Dog NPRA |
| 6 | ANP₂₈-20aa-CalgGB-Fc | 8760-110.3 | 3.0 +/− 0.9 |
| | | | (n = 3) |
| 6 | ANP₂₈-20aa-CalgGB-Fc | 8760-137 | 2.5 +/− 0.7 |
| | | | (n = 3) |
| 6 | ANP₂₈-20aa-CalgGB-Fc | 8760-174 | 5.4 +/− 1.0 |
| | | | (n = 3) |
| 6 | ANP₂₈-20aa-CalgGB-Fc | 9081-51 | 8.3 +/− 1.7 |
| | | | (n = 3) |
| 6 | ANP₂₈-20aa-CalgGB-Fc | 9081-53 | 9.4 +/− 3 |
| | | | (n = 3) |

PK studies in rats showed that these proteins had an increased half life in vivo.

### Example 5

### Preliminary PKPD Assessment of the Canine Fusion Protein in Dogs

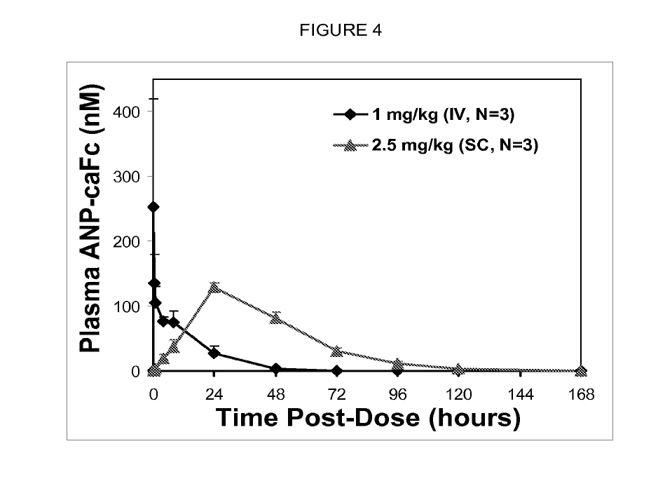

Dogs were administered a single IV dose (1 mg/kg or 17 nmol/kg) or SC dose (2.5 mg/kg or 42 nmol/kg) of ANP-caFc to assess PKPD over a period of 4 to 7 days. These doses were selected because they were similar to the dose-range employed to evaluate Cardeva (recombinant human serum albumin-BNP fusion protein) in dogs. FIG. 4 shows an estimate of the plasma ANP-caFc levels achieved in this study. The sensitivity of the ELISA used to reliably detect ANP-caFc in dog plasma was limited by having a minimum detection level in dog plasma of ˜50 nM. In order to avoid plasma matrix effects, a minimum dilution of 1:50 was required when assaying beagle plasma. The linear range of the assay, in PBS, is 100 μM to approximately 100 nM. In 2% beagle plasma the linear range of the curve is 1 nM to about 80 or 90 nM. FIG. 4 only provides an estimate of the estimated plasma levels and Table 2 presents the pharmacokinetics of ANP-caFc.

TABLE 2
Pharmacokinetics of ANP-caFc in Conscious
Dogs Following a Single Dose.

| | AUC | CLp | Vss | T½ | MRT |
|---|---|---|---|---|---|
| ROUTE | (μM · hr) | (mL/hr/kg) | (mL/kg) | (hr) | (hr) |
| IV | 2.0 ± 0.46 | 9.0 ± 2.3 | 107 ± 13.0 | 8.3 ± 2.0 | 12.2 ± 1.9 |
| SC | 6.1 ± 0.3 | | | 15.4 ± 1.8 | 41.6 ± 1.0 |

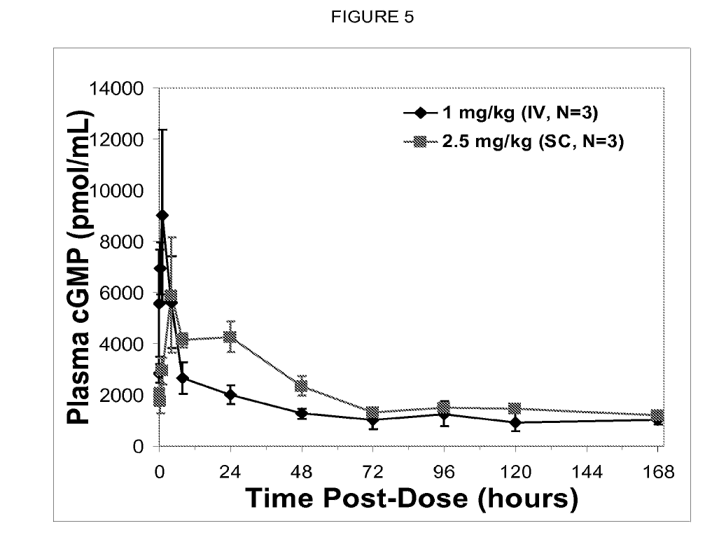

Plasma cGMP following SC dosing appeared to be elevated above baseline ≧48 hours post-dose (FIG. 5).

### Example 6

### Hemodynamic Assessment of the Canine Fusion Protein in Telemeterized Dogs

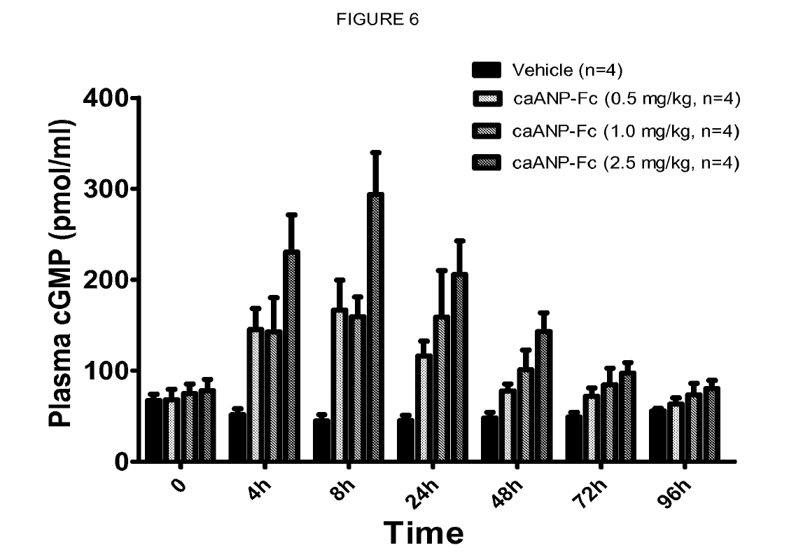

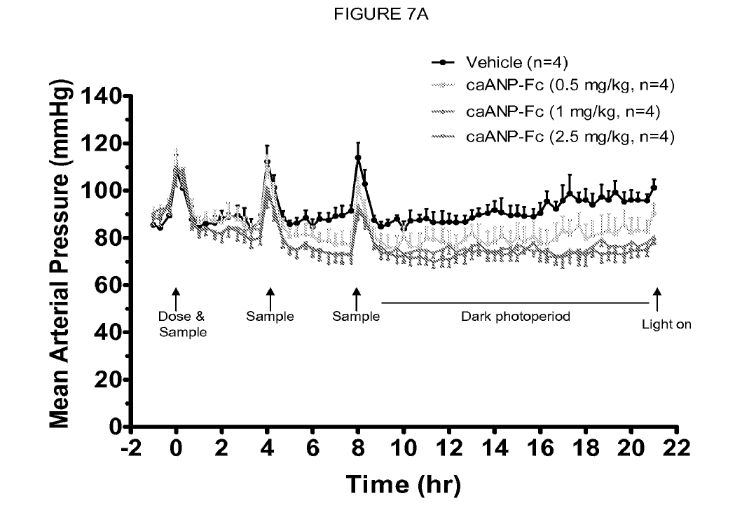

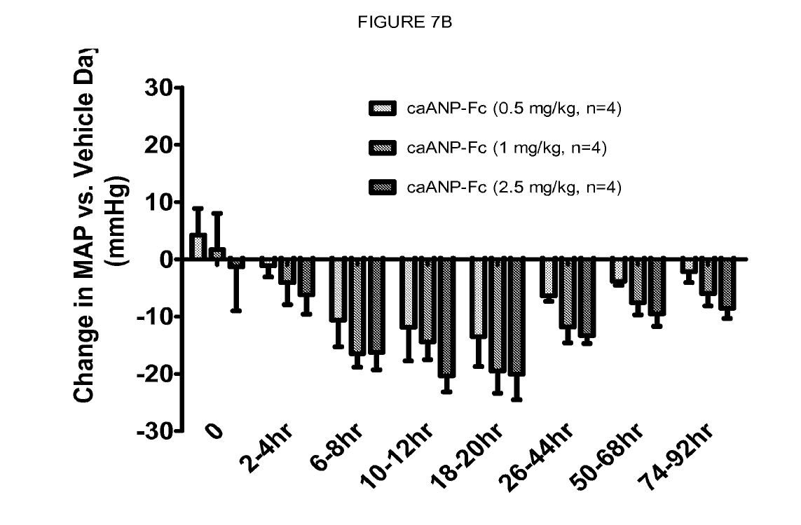

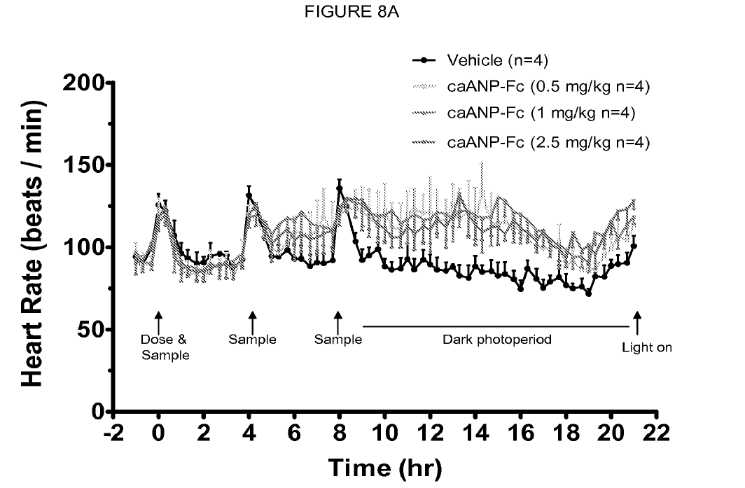

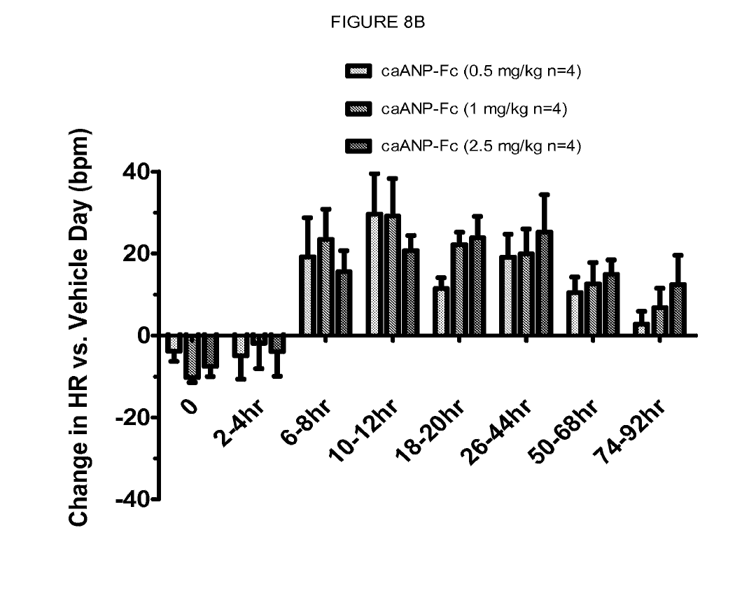

A study was performed in telemeterized conscious dogs to identify a dose of ANP-caFc that could be used to demonstrate efficacy in a canine heart failure model. The criteria for dose selection was that the dose could not decrease mean arterial blood pressure more than 5-10 mmHg below baseline, and that it should increase plasma cGMP concentration approximately 1.5×-2× above baseline. The first criterion was established based on achieving a desired clinical profile, i.e., that efficacy could be demonstrated without an arterial blood pressure reduction that would place some heart failure patients potentially at risk. The 1.5×-2× increase in plasma cGMP was based on work done in paced dogs using canine BNP which demonstrated a favorable cardiorenal effect at a dose which produced this magnitude of plasma cGMP elevation (H-H Chen et al. JACC 2000; 36:1706-12). In study #1, a total of 5 dogs were dosed subcutaneously with vehicle, or ANP-caFc at 0.5, 1 and 2.5 mg/kg based on a modified Latin square design over a 4-week period. This dose-range was chosen because it was expected to overlap the targeted plasma cGMP elevation based on the single dose dog PK study. The dosing interval was 7 days. One animal was excluded from the final data analysis due to a failure of telemetry device during the study. ANP-caFc produced a dose-dependent increase in peak plasma levels of cGMP (3.7-, 3.5- and 6.6-fold—of vehicle day value FIG. 6), a dose-dependent peak reduction in mean arterial blood pressure (MAP; −13±5, −19±4, and −20±5 mmHg vs. vehicle—FIGS. 7A and 7B), and peak heart rate was increased (+30 ±10, +29 ±9, and +25 ±9 beats per minute—FIGS. 8A and 8B).

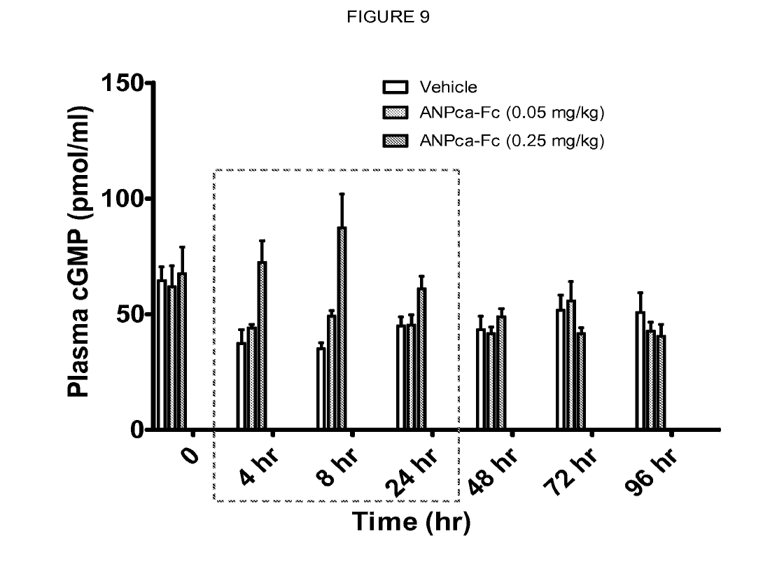

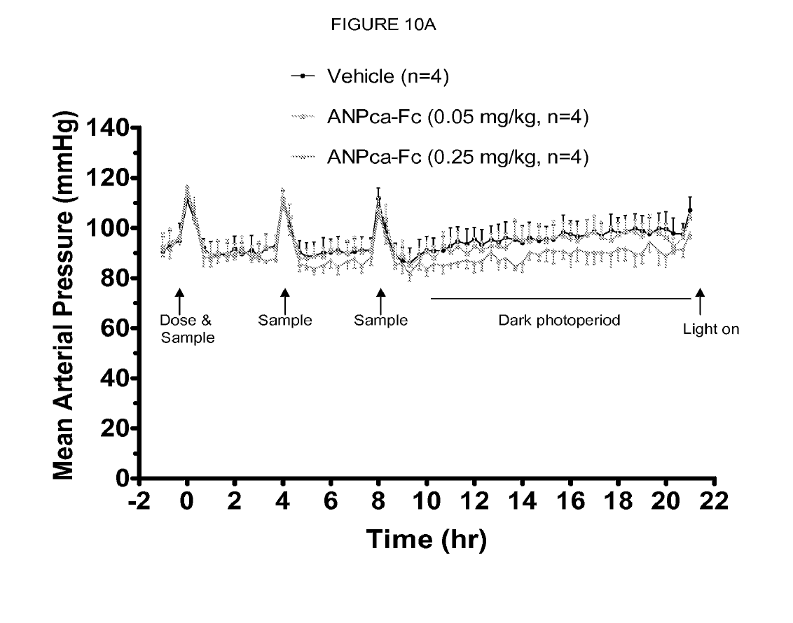

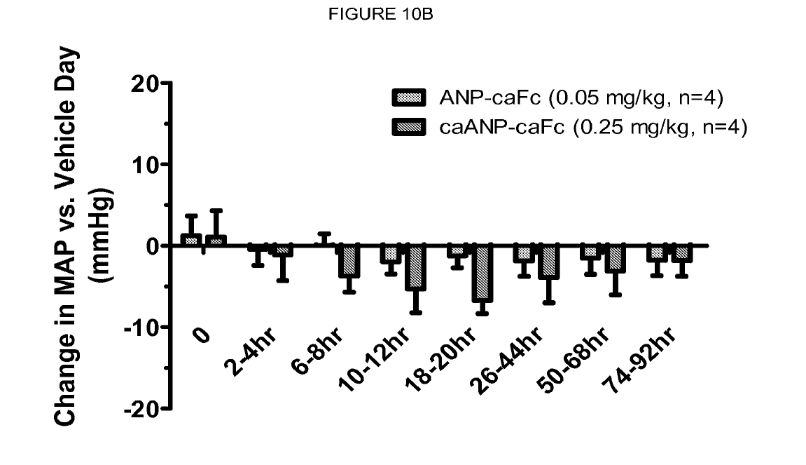

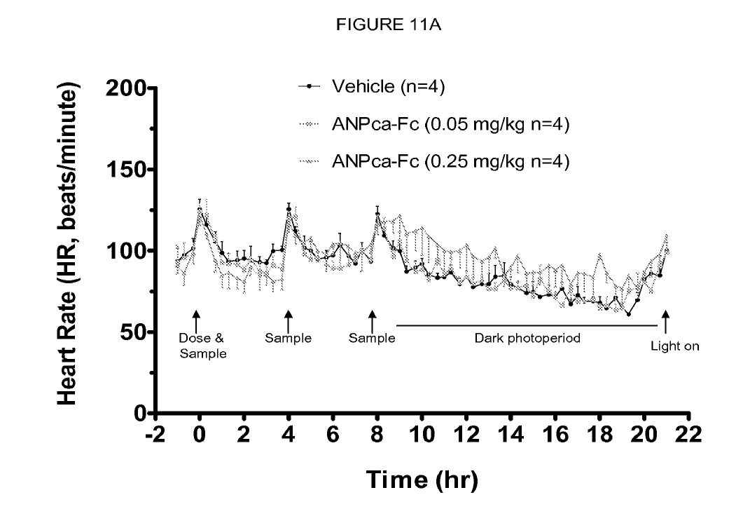

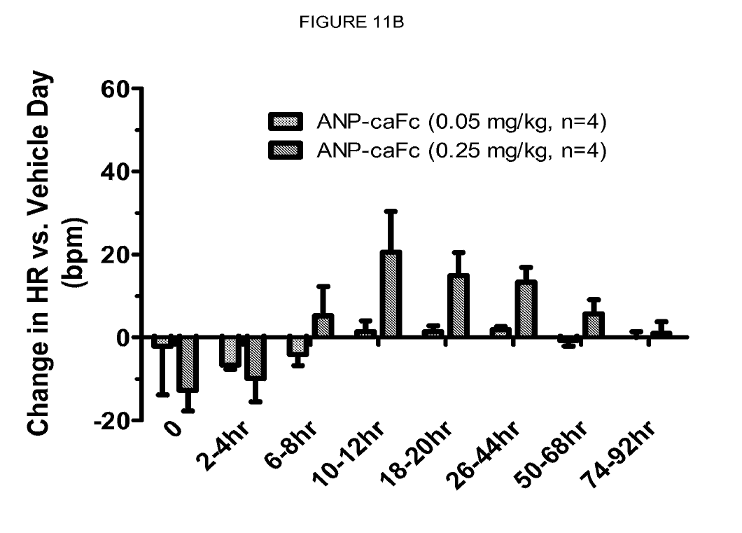

Since the MAP decreased more than 10 mmHg during the peak effect for all three doses, Study #2 was performed with lower doses (0.05 and 0.25 mg/kg) in the same 4 dogs using a similar study design. Study #2 revealed that the 0.25 mg/kg ANP-caFc dose produced a threshold effect for decreasing MAP (−6.6±1.6 mmHg vs. vehicle—FIG. 10), increasing heart rate (20±9 beats per minute—FIG. 11), and a moderate increase in plasma cGMP (FIG. 9). The 0.05 mg/kg dose of ANP-caFc had no effect of MAP (1.9±1.4 mmHg), HR (1.8±0.6 beats per minute), or plasma cGMP. (Urine samples were also collected for cGMP excretion measurements, however, samples were contaminated by drinking water and could not be used.)

1. A fusion protein comprising a therapeutic peptide or protein and a canine antibody Fc domain wherein the therapeutic peptide or protein is linked to the Fc domain directly or through a linker, wherein the Fc domain comprises a hinge region having a sequence selected from the group consisting of the hinge region of a canine IgG selected from the group consisting of canine IgGA, canine IgGB, canine IgGC and canine IgGD.

2. The fusion protein of claim 1, wherein the fusion protein comprises the following formula:
X-La-F:F-La-X or X-La-F:F, wherein,
X is a therapeutic peptide or protein;
L is a linker comprising a amino acid residues; a is an integer of at least 0;
“:” is a chemical association or crosslink; and
F is at least a portion of a canine immunoglobulin Fc domain comprising an FcRn binding site and comprises a hinge region selected from an canine IgGA, canine IgGB, canine IgGC and canine IgGD.

3. The fusion protein of claim 1 or claim 2 wherein the hinge region comprises a sequence selected from the group consisting of CTDTPPCP (SEQ ID NO:18); CPKCP (SEQ ID NO:19); FNECRCTDTPPCP (SEQ ID NO:20); PKRENGRVPRPPDCPKCP (SEQ ID NO:21); AKECECKCNCNNCPCPGCGL (SEQ ID NO:22); and PKESTCKCISPCP (SEQ ID NO:23).

4. The fusion protein of claim 1, wherein the therapeutic peptide is a natiuretic peptide selected from the group consisting of ANP, BNP, Urodilatin, DNP or a biologically active sequence variant thereof.

5. The fusion protein of claim 2, wherein the therapeutic peptide is ANP or BNP.

6. The fusion protein of claim 1, wherein the fusion protein comprises at least two therapeutic peptides.

7. The fusion protein of claim 6, wherein both therapeutic peptides are the same.

8. The fusion protein of claim 6, wherein at least one of peptides is a natiuretic peptide.

9. The fusion protein of claim 8 wherein said natriuretic peptide comprises a sequence of SEQ ID NO:8.

10. The fusion protein of claim 1, wherein the fusion protein comprises at least two Fc domains.

11. The fusion protein of claim 1, wherein the linker is 6 amino acids in length, 11 amino acids in length, 16 amino acids in length or 20 amino acids in length.

12. The fusion protein of claim 1, wherein the linker is 6 to 11 amino acids in length, 11 to 16 amino acids in length, 16 to 20 amino acids in length, 16 to 25 amino acids in length or 20 to 30 amino acids in length.

13. The fusion protein of claim 1, wherein the linker is a glycine succinate linker, an amino acid linker or combination thereof.

14. The fusion protein of claim 1, wherein the amino acid linker is GlyGly (L2), Gly(SerGlyGly)2SerGly (L3) (SEQ ID NO. 13), (GlyGlySer)3 GlyGly (L4) (SEQ ID NO. 14), (GlyGlySer)4GlyGly (SEQ ID NO. 15), (GlySerGly)5Gly (L5a) (SEQ ID NO. 16), (GlyGlySer)5Gly (L5) (SEQ ID NO. 17), or (GlyGlySer)6GlyGly (L6) (SEQ ID NO:12).

15. A fusion protein comprising at least one or more therapeutic peptide separated from each other by a canine antibody Fc domain that comprises a hinge sequence selected from the group consisting of CTDTPPCP; CPKCP (SEQ ID NO:19); FNECRCTDTPPCP (SEQ ID NO:20); PKRENGRVPRPPDCPKCP (SEQ ID NO:21); AKECECKCNCNNCPCPGCGL (SEQ ID NO:22); and PKESTCKCISPCP (SEQ ID NO:23), wherein said thereapeutic peptides are conjugated to the Fc domain directly or through a linker.

16. The fusion protein of claim 15, wherein the fusion protein comprises the following formula:
X-La-F:F-La-X, wherein
X is one or more therapeutic peptides or proteins
L is a linker comprising amino acid residues; a is an integer of at least 0;
“:” is a chemical association or crosslink; and
F is at least a portion of an immunoglobulin Fc domain comprising an FcRn binding site.

17. The fusion peptide of claim 16 wherein at least one of the therapeutic peptides has a sequence of SEQ ID NO:8.

18. The fusion protein of claim 16, wherein X is more than one natriuretic peptide.

19. An isolated fusion protein having the sequence of SEQ ID NO:2, SEQ ID NO:3; SEQ ID NO:5 or SEQ ID NO:6 or an isolated fusion protein that exhibits at least 99% sequence identity with a polypeptide having a sequence selected from the group consisting of SEQ ID NO. 2, SEQ ID NO. 3, SEQ ID NO. 5 and SEQ ID NO. 6.

20. (canceled)

21. An isolated nucleic acid molecule encoding a polypeptide comprising amino acid sequences selected from the group consisting of SEQ ID NO. 2, SEQ ID NO. 3, SEQ ID NO. 5 and SEQ ID NO. 6.

22. An isolated nucleic acid molecule encoding a polypeptide comprising amino acid sequences selected from the group consisting of SEQ ID NO. 1 and SEQ ID NO. 4.

23. The fusion protein of claim 1, wherein said fusion protein is recombinantly produced by employing mammalian, prokaryotic, yeast, plant, or transgenic expression systems.

24. A pharmaceutical composition comprising a fusion protein of claim 1.

25. The pharmaceutical composition of claim 24, wherein the fusion protein is adapted for intravenous, subcutaneous or oral administration.

26. The pharmaceutical composition of claim 24, wherein the fusion protein is adapted for intravenous administration.

27. A method of treating or ameliorating a condition characterized by an excessive level of extracellular fluid, the method comprising administering to a subject in need thereof a therapeutically effective amount of the pharmaceutical composition of claim 24.

28. A method of treating or ameliorating a pathological condition in which activation of the NPRA receptor confers a therapeutic benefit comprising administering to a subject in need thereof a therapeutically effective amount of the pharmaceutical composition of claim 24.

29. A method of treating or ameliorating a disease associated with abnormal diruretic, natriuretic and vasodilatory activity comprising administering to a subject in need thereof a therapeutically effective amount of the pharmaceutical composition of claim 24.

30. A method of treating or ameliorating a disease in which it is desirable to induce naturesis, diuresis, vasodilation or to modulate the renin-angiotensin II and aldosterone systems comprising administering to a subject in need thereof a therapeutically effective amount of the pharmaceutical composition of claim 24.

31. A method to treat or ameliorate a pathological condition of the cardiovascular system selected from the group consisting of chronic heart failure (non-ischemic), reperfusion injury, left ventricular dysfunction (LVD), cardiac fibrosis, diastolic heart failure, and hypertrophic cardiomyopathy comprising administering to a subject in need thereof a therapeutically effective amount of the pharmaceutical composition of claim 24.

32. A method to treat or ameliorate a hypertensive disorder selected from the group consisting of hypertension, pulmonary hypertension, systolic hypertension and resistant hypertension comprising administering to a subject in need thereof a therapeutically effective amount of the pharmaceutical composition of claim 24.

33. A method to treat or ameliorate diabetic nephropathy comprising administering to a subject in need thereof a therapeutically effective amount of the pharmaceutical composition of claim 24.
